SEQUENCE LISTING (11 sequences)

SEQ ID NO: 1 - protein, 242 aa, Artificial sequence
  Synthetic sequence
AAAKPATTEGEFPETREKMSGIRRAIAKAMVHSKHTAPHVTLMDEADVTKLVAHRKKFKAIAAEKGIKLTFLPYVVKALVSALREYPVLNTAIDDETEEIIQKHYYNIGIAADTDRGLLVPVIKHADRKPIFALAQEINELAEKARDGKLTPGEMKGASCTITNIGSAGGQWFTPVINHPEVAILGIGRIAEKPIVRDGEIVAAPMLALSLSFDHRMIDGATAQKALNHIKRLLSDPELLLM

SEQ ID NO: 2 - protein, 205 aa, Artificial sequence
  Synthetic sequence
MKMEELFKKHKIVAVLRANSVEEAKMKALAVFVGGVHLIEITFTVPDADTVIKELSFLKELGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTIAEVAAKAAAFVEKIRGCTE

SEQ ID NO: 3 - protein, 5 aa, Artificial sequence
  Synthetic sequence
GGGGS

SEQ ID NO: 4 - protein, 625 aa, Artificial sequence
  Synthetic sequence
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVMIRSENITNNAKNILVQFNTPVQINCTRPNNNTRKSIRIGPGQAFYATGDIIGDIRQAHCNVSKATWNETLGKVVKQLRKHFGNNTIIRFANSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGGGGGSGGGGSAVGIGAVFLGFLGAAGSTMGAASMTLTVQARNLLSGNPDWLPDMTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 5 - protein, 58 aa, Artificial sequence
  Synthetic sequence
FSEEQKKALDLAFYFDRRLTPEWRRYLSQRLGLNEEQIERWFRRKEQQIGWSHPQFEK

SEQ ID NO: 6 - protein, 46 aa, Artificial sequence
  Synthetic sequence
SPAVDIGDRLDELEKALEALSAEDGHDDVGQRLESLLRRWNSRRAD

SEQ ID NO: 7 - protein, 13 aa, Artificial sequence
  Synthetic sequence
AKFVAAWTLKAAA

SEQ ID NO: 8 - protein, 10 aa, Artificial sequence
  Synthetic sequence
GGGGSGGGGS

SEQ ID NO: 9 - protein, 8 aa, Artificial sequence
  Synthetic sequence
NPDWLPDM

SEQ ID NO: 10 - protein, 4 aa, Artificial sequence
  Synthetic sequence
REKR

SEQ ID NO: 11 - protein, 6 aa, Artificial sequence
  Synthetic sequence
RRRRRR

(19) United States
(12) Patent Application Publication
(10) Pub. No.: US 2023/0181718 A1
(43) Pub. Date: Jun. 15, 2023

(54) METHODS AND COMPOSITIONS RELATED TO VIRAL VACCINES WITH IMPROVED PROPERTIES

(71) Applicant: THE SCRIPPS RESEARCH INSTITUTE, La Jolla, CA (US)

(72) Inventors: Linling He, San Diego, CA (US); Jiang Zhu, San Diego, CA (US)

(21) Appl. No.: 17/743,762

(22) Filed: May 13, 2022

Related U.S. Application Data
(63) Continuation-in-part of application No. 17/550,768, filed on Dec. 14, 2021.

(51) Int. Cl. A61K 39/21; A61K 39/145; A61K 39/215; A61K 39/29; A61K 39/12; A61P 31/18; C12P 21/00

(52) U.S. Cl. CPC A61K 39/21; A61K 39/145; A61K 39/215; A61K 39/29; A61K 39/12; A61P 31/18; C12P 21/00; C12Y 302/01096

(57) ABSTRACT

The present invention provides methods for producing scaffolded (e.g., nanoparticle presented) or non-scaffolded vaccines that are based on viral immunogenic proteins with a glycan shielded (e.g., HIV-1 Env). The vaccines thus generated demonstrate enhanced immunogenicity and responder frequency. The methods entail (1) enzymatic digestion of glycan chain on the surface of a viral trimer protein immunogen or a self-assembling nanoparticle vaccine displaying the viral immunogen (e.g., an HIV-1 UFO trimer), or (2) expression of a construct encoding the vaccine in an expression system lacking normal glycosylation function for human proteins. Also provided in the invention are vaccine compositions produced with the described methods. The invention further provides methods of using the vaccine compositions described herein (e.g., scaffolded or non-scaffolded HIV-1 vaccines) in various therapeutic applications, e.g., for preventing or treating viral infections.

### CROSS-REFERENCE TO RELATED APPLICATIONS

The subject patent application is a continuation-in-part of U.S. patent application Ser. No. 17/550,768 (filed Dec. 14, 2021; now pending). The full disclosure of the priority application is incorporated herein by reference in its entirety and for all purposes.

### STATEMENT OF GOVERNMENT SUPPORT

This invention was made with government support under Grant Number AI129698 awarded by the National Institutes of Health. The government has certain rights in the invention.

### BACKGROUND OF THE INVENTION

HIV-1 envelope glycoprotein (Env) is the sole target for neutralizing antibodies (NAbs) during natural infection. Like other class-I viral fusion proteins, HIV-1 Env is a homotrimer of gp120-gp41 heterodimers. Before infection, the trimeric Env proteins on HIV-1 virions are in the prefusion state, which is intrinsically unstable (or termed metastable) in order to undergo a rapid conformational change to mediate cell entry. In a small fraction (5-15%) of HIV-1 patients, some NAbs generated during early infection can co-evolve with viruses and become broadly neutralizing antibodies (bNAbs) over an extended period of time (e.g., more than ten years). So far, panels of bNAbs have been isolated from elite HIV-1 patients, suggesting that protective HIV-1 vaccines may be achieved by eliciting such bNAbs during immunization. Significant progresses in Env stabilization have been made in rational HIV-1 vaccine design in the recent years. Various design strategies were proposed to stabilize HIV-1 Env in “native-like” prefusion trimer structures (Ward & Wilson, Immunol Rev 2017, 275(1):21-32; Sanders & Moore, Immunol Rev 2017, 275(1):161-182). Notably, one strategy based on the analysis of Env metastability proved to be highly effective and provided a general solution to Env-based HIV-1 vaccine design. First, an HR1 bend (aa 547-569) in the gp41 ectodomain (gp41_{ECTO}) was identified as the primary cause of HIV-1 Env metastability (Kong et al., Nat. Commun. 2016, 7:12040). An uncleaved, prefusion-optimized (UFO) design was developed to stabilize HIV-1 Env trimers by combining a redesigned HR1 bend and a GS linker at the furin cleavage site. The UFO design has been applied to diverse HIV-1 Envs with great success, producing soluble trimers with significantly higher yield, purity, and stability than the SOSIP and NFL designs. It was also demonstrated that gp41_{ECTO} is the sole source of metastability and BG505 gp41_{ECTO} of the UFO design (termed UFO-BG) can be used to stabilize diverse HIV-1 Envs with substantial trimer yield, purity, and stability (He et al., Sci Adv 2018, 4:aau6769). The UFO and UFO-BG designs thus provide a simple, general, and effective strategy for Env stabilization and trimer-based HIV-1 vaccine design. The BG505 SOSIP trimer has been tested in mice with different adjuvants and injection routes and using osmotic pumps but failed to induce any tier 2 neutralizing antibody response (Hu et al., J. Virol. 89: 10383-98, 2015). In rabbits, both SOSIP and NFL trimers induced tier 2 neutralizing antibody responses that were mainly directed to the glycan holes on the Env. In non-human primates (NHPs), SOSIP and NFL trimers only included sporadic tier-2 neutralizing antibody responses. In another study (He et al., Sci. Adv. 4:aau6769, 2018), thirteen HR1-redesigned BG505 trimers were tested in mice, and no tier 2 neutralizing antibody response was observed. Thus, despite the advancements in HIV-1 trimer design, elicitation of tier 2 neutralizing antibody response remains a challenge for soluble trimer-based vaccines.

It is well known that single-subunit vaccines are less effective than virus-like particles (VLPs), which induce strong immune responses due to their large size and dense antigen display. VLPs have shown exceptional success as human vaccines, as exemplified by vaccines developed for human papillomaviruses (HPV), hepatitis B virus (HBV), and hepatitis E virus (REV). Self-assembling protein nanoparticles (SApNPs) have been considered an alternative for developing VLP vaccines without involving complicated purification methods typically required for VLPs. Development of SApNP vaccines for HIV-1 and other viral pathogens have been reported. These include, e.g., display of trimeric HIV-1 antigens such as V1V2, gp120, and gp140 on 24-meric ferritin and 60-meric E2p (He et al., Nat. Commun. 2016, 7:12041), and display of UFO and UFO-BG trimers of diverse HIV-1 Envs on 24-meric ferritin and 60-meric 13-01 (He et al., Sci Adv 2018, 4:aau6769).

Despite the substantial progresses in vaccine design, major issues remain to be addressed in the development of potent and effective HIV-1 vaccines, e.g., low immunogenicity and sporadic tier 2 neutralizing antibody responses, the latter of which can be described as the low frequency of vaccine responders. The present invention is intended to address these and other unmet needs in the art.

### SUMMARY OF THE INVENTION

In one aspect, the invention provides methods for enhancing immunogenicity or responder frequency of a vaccine that contains a glycan shielded immunogenic protein or a variant thereof from a virus. The methods entail (a) contacting the vaccine with an enzyme that is capable of removing or shortening the N-linked glycan chain from the protein, or (b) expressing a polynucleotide sequence encoding the protein or variant thereof in a cell line that produces short glycans and/or lacks N-acetylglucosaminyltransferase I. These treatments lead to enhanced immunogenicity and frequency of vaccine responders. In various embodiments, the glycan shielded viral immunogenic protein or variant in the vaccine is from HIV-1, Lassa virus, hepatitis C virus, influenza virus, or SARS-CoV-2. In some methods, the entire length of the N-linked glycan chain on the protein is trimmed. In some other methods, the length of the N-linked glycan chain is substantially trimmed, e.g., by at least about 50%. In various embodiments, the employed enzyme for trimming the glycan chain is endoglycosidase H (Endo-H), F1 (Endo-F1), F2 (Endo-F2), or F3 (Endo-F3).

Some of the methods are directed to HIV-1 vaccines that contain a modified HIV-1 envelope gp140 protein, but not a HIV-1 gp160 protein or just a HIV-1 gp120 protein alone. The modified HIV-1 Env gp140 protein is comprised of a gp120 polypeptide (or fragment or variant thereof), and a gp41 polypeptide that has its N-terminus of heptad 1 region (HR1) replaced with a loop sequence of about 6 to about 14 amino acid residues in length that stabilizes the pre-fusion gp140 structure. In some preferred embodiments, the modified HIV-1 envelope gp140 protein is an uncleaved prefusion-optimized (UFO) gp140 trimer. In some of these embodiments, the modified HIV-1 envelope gp140 protein is a chimeric trimer comprising a modified gp41_{ECTO} domain from HIV-1 strain BG505. In some embodiments, the modified HIV-1 envelope gp140 protein further contains a flexible linker sequence that substitutes for the cleavage site sequence between gp120 and gp41. In some of these embodiments, a linker sequence (G₄S)₂ (SEQ ID NO:8) substitutes for residues 501-518 at the cleavage site, and the numbering of the amino acid residues corresponds to that of HIV-1 strain BG505 SOSIP.664 gp140. In various embodiments, the modified HIV-1 envelope gp140 protein can additionally contain (a) an engineered disulfide bond between gp120 and gp41, and/or (b) a stabilizing mutation in gp41. For example, the BG505 SOSIP.664 gp140 protein has an engineered disulfide bond between residues A501C and T605C, and a stabilizing mutation I559P.

In some exemplified methods, the modified HIV-1 Env gp140 protein is derived from BG505 SOSIP.664 gp140. It contains a gp41_{ECTO} polypeptide that has its N-terminus of heptad 1 region (HR1) replaced with a loop sequence NPDWLPDM (SEQ ID NO:9). In some of these embodiments, the modified HIV-1 Env gp140 protein can additionally contain (a) a linker sequence (G₄S)₂ (SEQ ID NO:8) that substitutes for residues 508-511 at the cleavage site, and (b) an engineered disulfide bond between residues A501C and T605C. As a specific exemplification, the employed vaccine contains an HIV-1 UFO trimer protein with a sequence set forth in SEQ ID NO:4, or a substantially identical or conservatively modified variant sequence.

In various embodiments, the vaccine material can be treated with the enzyme under appropriate conditions to allow thorough digestion of the glycan chain, e.g., with Endo-H at room temperature (e.g., 25° C.) without denaturing for a sufficient period of time (e.g., 1, 2, 3, 4, 5, 6 or more hours). In some embodiments, the employed enzyme vs protein ratio is sufficient for complete enzymatic digestion of N-linked glycans on the immunogenic protein or variant thereof. Some methods of the invention further contain a step of purification of the enzyme treated vaccine.

In a related aspect, the invention provides methods for enhancing immunogenicity of HIV-1 nanoparticle vaccines. The methods involve (a) contacting an HIV-1 nanoparticle vaccine with an enzyme that is capable of removing or shortening the N-linked glycan chain from the vaccine polypeptide sequence, or (b) expressing a polynucleotide sequence encoding the subunit of the HIV-1 nanoparticle vaccine in a cell line expression system that produces shorter Man₃₋₅GlcNAc₂ glycans and/or lacks N-acetylglucosaminyltransferase I. In some embodiments, the entire length of the N-linked glycan chain on the HIV-1 vaccine polypeptide is trimmed. In some other embodiments, the length of the N-linked glycan chain is trimmed by about 50%, about 60%, about 70%, about 80%, or about 90%.

In some methods of the invention, the enzyme used for trimming the glycan chain is an endoglycosidase (Endo) or a peptide/N-glycosidase (PNGase). In some preferred embodiments, the employed enzyme is endoglycosidase H, F1, F2, or F3 (Endo-H, F1, F2, or F3). In some of these embodiments, the HIV-1 nanoparticle vaccine is contacted with the enzyme at room temperature, using an expressed and purified HIV-1 SApNP vaccine. In these methods, the employed HIV-1 vaccine material (protein) is preferably not denatured. In these embodiments, the enzymatic digestion can last for 1 hr, 2 hrs, 3 hrs, 4 hrs, 5 hrs, 6 hrs, or even longer. In some of these embodiments, the employed endo-H vs SApNP ratio is 12,500 units vs. 100 μg to ensure complete trimming of N-linked glycans on the surface. In some methods of the invention, the enzyme trimmed HIV-1 SApNP vaccine is further subject to protein purification.

In some embodiments, the HIV-1 vaccine for glycan trimming contains a polypeptide sequence having from the N-terminus to the C-terminus (1) the subunit sequence of an HIV-1 Env-derived trimer, (2) the subunit sequence of a self-assembling nanoparticle, and (3) a locking domain subunit sequence. In some of these methods, the employed locking domain subunit sequence is fused to C-terminus of the nanoparticle subunit sequence via a linker sequence. In some embodiments, the linker sequence between the locking domain and the nanoparticle subunit contains GGGGS (SEQ ID NO:3). In some embodiments, the polypeptide sequence of the HIV-1 vaccine additionally contains a pan-reactive T-cell epitope that is fused to the C-terminus of the locking domain subunit sequence. In some of these embodiments, the employed T-cell epitope has the sequence AKFVAAWTLKAAA (SEQ ID NO:7). In some embodiments, the HIV-1 trimer subunit sequence is fused to the nanoparticle subunit sequence via a linker sequence. In some of these embodiments, the linker between the HIV-1 trimmer sequence and the nanoparticle sequence contains the sequence (GaSb)n, wherein a is an integer of 1 to 5, b is an integer of 1 to 2, and n is an integer of 1 to 5.

In various embodiments, the displaying nanoparticle scaffold of the HIV-1 vaccine contains a trimeric sequence. In some of these embodiments, the subunit sequence of the self-assembling nanoparticle contains SEQ ID NO:1 (E2p) or SEQ ID NO:2 (I3-01 variant), a conservatively modified variant or a substantially identical sequence thereof. In some embodiments, the employed HIV-1 nanoparticle vaccine displays an uncleaved prefusion-optimized (UFO) HIV-1 gp140 trimer. In some of these embodiments, the UFO gp140 trimer is a chimeric trimer containing a modified gp41_{ECTO} domain from HIV-1 strain BG505. In some of these embodiments, the subunit sequence of the UFO gp140 trimer contains the sequence shown in SEQ ID NO:4, a conservatively modified variant or a substantially identical sequence thereof.

In some embodiments, the polypeptide sequence from which the HIV-1 vaccine is formed contains from the N-terminus to the C-terminus: HIV-1 Env-derived UFO gp140 trimer subunit as shown in SEQ ID NO:4, self-assembling nanoparticle subunit as shown in SEQ ID NO:1 (E2p), the locking domain as shown in SEQ ID NO:5 (LD4), and T-cell epitope AKFVAAWTLKAAA (SEQ ID NO:7). In some of these embodiments, the polypeptide sequence further contains a first linker sequence (GGGGS)₂ (SEQ ID NO:8) between the gp140 trimer subunit and the nanoparticle subunit, and/or a second linker sequence GGGGS (SEQ ID NO:3) between the nanoparticle subunit and the locking domain. In some other embodiments, the polypeptide sequence from which the HIV-1 vaccine is formed contains from the N-terminus to the C-terminus: HIV-1 Env-derived UFO gp140 trimer as shown in SEQ ID NO:4, self-assembling nanoparticle subunit as shown in SEQ ID NO:2 (13-01 variant), the locking domain as shown in SEQ ID NO:6 (LD7), and T-cell epitope AKFVAAWTLKAAA (SEQ ID NO:7). In some of these embodiments, the polypeptide sequence further contains a first linker sequence (GGGGS)₂ (SEQ ID NO:8) between the gp140 trimer subunit and the nanoparticle subunit, and/or a second linker sequence GGGGS (SEQ ID NO:3) between the nanoparticle subunit and the locking domain.

In some methods of the invention, glycan trimming is achieved by the expressing the HIV-1 vaccine construct in 519 insect cell or HEK293F GnTI− cells.

In a related aspect, the invention provides HIV-1 Env trimer vaccines that are produced by a process containing the steps of: (1) expressing a polynucleotide encoding an HIV-1 Env immunogenic protein to generate an HIV-1 vaccine, and (2) trimming N-glycosylation chain on the expressed immunogenic protein with an enzyme. In some embodiments, the enzyme employed for trimming the glycan chain is endoglycosidase H, F1, F2, or F3 (Endo-H, F1, F2, or F3). In some of these embodiments, the process further contains the step of purification of the expressed vaccine prior to the glycan trimming. In some related embodiments, the invention provides pharmaceutical compositions that contain a glycan-trimmed HIV-1 Env trimer vaccine described herein, and a pharmaceutically acceptable carrier. In a related aspect, the invention provides methods for treating or preventing HIV-1 infection in a subject. The methods entail administering to a human subject in need of treatment the pharmaceutical composition of the invention.

A further understanding of the nature and advantages of the present invention may be realized by reference to the remaining portions of the specification and claims.

### DESCRIPTION OF THE DRAWINGS

FIG. 1 shows results from biochemical characterization of Endo-H-treated CHO-K1-produced HIV-1 SApNPs. A: SEC profiles from Superose 6 column purification of treated vaccine protein. B: Analysis with reducing SDS-PAGE.

FIG. 2 shows site-specific glycan profiles two CHO-K1-produced HIV-1 SApNPs using liquid chromatography-mass spectrometry (LC-MS).

FIG. 3 shows negative stain EM analysis of glycan-trimmed HIV-1 SApNPs.

FIG. 4 shows bio-layer interferometry (BLI) analysis HIV-1 SApNPs with/without glycan trimming.

FIG. 5 shows neutralization of titer-2 BG505.T332N by purified mouse IgG from week 11 after 2 primes and 2 boosts. Two HIV-1 SApNPs are tested in mouse immunization with two different prime/boost regimens.

FIG. 6 shows biochemical characterization of Endo-H treated BG505 UFO.664 trimer.

FIG. 7 shows removal of residual Endo-Hf and assessment of trimer purify.

FIG. 8 shows negative-stain EM analysis of glycan-trimmed BG505 UFO.664 trimer with bNAbs.

FIG. 9 provides a summary of peak Env-Antibody binding signals from Bio-layer interferometry (BLI) analysis of the BG505 UFO.664 trimer.

FIG. 10 shows neutralization against tier-2 BG505.T332N by w8 mouse serum after 2 primers and 1 boost.

### DETAILED DESCRIPTION

### I Overview of the Invention

A main bottleneck of HIV-1 vaccine development is the low immunogenicity of the vaccines in eliciting neutralizing antibody response in vivo. In a 2015 study, the BG505 SOSIP trimer was tested thoroughly in wildtype mice with different adjuvants, immunization routes, and slow-release devices (e.g., osmotic pumps), but failed to induce any detectable tier-2 neutralizing antibody response (Hu et al., J. Virol. 2015, 89: 10383-10398). In rabbits, both SOSIP and NFL trimers induced tier-2 neutralizing antibody responses that mainly target the glycan holes on the Env. In non-human primates (NHPs), these stable trimers only induced sporadic tier-2 neutralizing antibody responses. It was previously demonstrated that trimer-presenting SApNPs can induce a potent tier-2 neutralizing antibody response in wildtype mice and rabbits (He et al., Sci Adv 2018, 4:aau6769). However, despite the advancements attributed to rational trimer design and nanoparticle display, tier-2 neutralizing antibody responses are only sporadic, with most animals showing negligible or no neutralizing antibody response.

HIV-1 Env is heavily glycosylated, with ˜30 glycans per protomer and ˜90 glycans per trimer, which contribute to ˜50% of the mass and form a dense glycan shield on the surface. Broadly neutralizing antibodies (bNAbs) isolated from elite patients can be categorized based on their epitopes, including the trimeric V2 apex, V3 stem, CD4-binding site (CD4bs), gp120-gp41 interface, and the membrane-proximal external region (MPER). The V2 apex- and V3 stem-directed antibodies constitute a large portion of serum response in the cohort analysis of HIV-1 patients who have developed bNAbs. Notably, a hallmark of the V2 apex- and V3 stem-directed bNAbs is that they must interact with N-linked glycans at specific positions within or around the epitope. For example, the V2-directed bNAbs, such as PG9, PG16, and PGT145, require a Man₈₋₉GlcNac₂ glycan structure at N160 for neutralization and may involve the hybrid-type glycans at N173 and N156. The V3 stem-directed bNAbs, such as PGT121, PGT124, PGT128, and PGT135, require a Man8-9GlycNac2 glycan structure at N332 for neutralization and may involve glycans at N295, N301, and other sites. Due to the involvement of multiple glycans, the V3 stem epitope is also called a glycan supersite. Therefore, altering or trimming glycans is generally thought to impair Env binding to glycan-reactive bNAbs, as demonstrated for the PG9 and PG16 antibodies (Doores et al., J. Virol. 84:10510-21, 2010). There are reports that glycan trimming improved immunogenicity of the soluble influenza hemagglutinin trimers. See, e.g., de Vries et al., J. Virol. 86:16735-44, 2012; and Wang et al., Proc. Natl. Acad. Sci. USA 106:18137-42, 2009. However, there has been no actual evidence or subsequent reports that glycan trimming would indeed improve activities of NP-displayed influenza vaccines, let alone HIV-1 NP vaccines. Instead, because N-linked glycans have been considered in the art an integral part of Env epitope structure (See, e.g., Crispin et al., Annu. Rev. Biophys, 2018), N-linked glycans have been exploited as an important target in HIV-1 vaccine design. See, e.g., Doores, FEBS J, 282: 467:9-91, 2015; and Wagh et al., Curr. Opin. HIV AIDS, 15:267-74, 2020. Consistently, one strategy that is gaining popularity in HIV-1 vaccine design is to increase Env glycan occupancy to mimic native viral spike. See, e.g., Derking et al, Cell Rep, 35:108933, 2021.

The present invention is predicated in part on the studies undertaken by the inventors to increase the frequency of vaccine-responders (FVR) after vaccination (e.g., from ˜10% to ˜50% or greater), which would allow the generation of more effective HIV-1 vaccines. The inventors explored various approaches and surprisingly discovered that, contrary to what the consensus view in HIV-1 vaccine field would strongly suggest, a simple “glycan-trimming” step during nanoparticle-based vaccine production process can dramatically increase the FVR of UFO trimer-presenting SApNPs, rendering them highly immunogenic. It was unexpectedly observed that glycan trimming by enzymatic treatment exhibited no adverse effect on the biochemical, biophysical, and antigenic properties of UFO trimer-presenting SApNPs. As detailed herein, HIV-1 UFO trimer-presenting SApNPs can be processed enzymatically to trim N-linked glycans on the Env trimers to expose the most conserved CD4 binding site (CD4bs) and other protein epitopes. Specifically, it was observed by the inventors that glycan trimming is independent of the nanoparticle platforms and Env backbones, as exemplified with HIV-1 BG505 UFO trimer presented on an E2p (SEQ ID NO:1) and an 13-01 variant (SEQ ID NO:2) nanoparticle scaffolds. The glycan trimming significantly increased the vaccine immunogenicity and induced potent neutralizing antibody responses. Importantly, it was observed that nearly identical in vitro outcomes were obtained with two UFO trimer vaccines based on different nanoparticle scaffolds, as evidenced by results from SEC, SDS-PAGE, ns-EM, and BLI. In mouse immunization, potent neutralizing antibody response was observed for both nanoparticle scaffolds after glycan trimming, and the frequency of vaccine responders (FVR) appeared to be notably increased for the 13-01 scaffold (SEQ ID NO:2). While only BG505 UFO trimer-presenting nanoparticle vaccines were exemplified in the studies, this glycan trimming approach could be equally effective for SApNP vaccines derived from non-BG505 Env backbones as Endo-H has been widely used to trim Env glycans for crystallization.

In a further study, the inventors assessed the effect of glycan trimming on the immunogenicity of vaccines containing non-scaffolded HIV-1 Env trimmer proteins. Specifically, HIV-1 UFO trimers were processed to shorten N-linked glycans with endoglycosidase H (Endo-H), which cleaves within the chitobiose core of high mannose and some hybrid oligosaccharides from N-linked glycoproteins. As detailed herein, it was found that, similar to the effect on nanoparticle presented HIV-1 Env trimers, glycan trimming also significantly increased immunogenicity of the non-scaffolded vaccine and induced potent neutralizing antibody responses in animal models.

The glycan-trimmed HIV-1 UFO trimer-presenting SApNPs and non-scaffolded trimers of the invention can be used as prime and/or boost immunogens in preventive HIV-1 vaccines to enhance immunogenicity and increase the frequency of vaccine responders. Glycan trimming by Endo-H can be easily included as an additional step in the down-stream process and expected to deliver materials with high purity, high structural homogeneity, consistent glycan profiles, and improved antigenicity, thus enabling large-scale industrial production in a GMP facility. Further, the same or similar enzymatic treatment protocols can also be used to enhance immunogenicity of vaccines based on envelope proteins from other glycan-shielded viruses. Thus, for example, glycan trimming can be readily employed in the preparation of scaffolded or non-scaffolded vaccines generated with the envelope protein of additional viruses such as Lassa viruses (LASV), hepatitis C virus (HCV), and SARS-CoV-2.

Unless otherwise specified herein, compositions and methods related to the glycan trimmed HIV-1 vaccines of the invention can all be generated or performed in accordance with the procedures exemplified herein or routinely practiced methods well known in the art. See, e.g., Methods in Enzymology, Volume 289: Solid-Phase Peptide Synthesis, J. N. Abelson, M. I. Simon, G. B. Fields (Editors), Academic Press; 1st edition (1997) (ISBN-13: 978-0121821906); U.S. Pat. Nos. 4,965,343, and 5,849,954; Sambrook et al., Molecular Cloning: A Laboratory Manual, Cold Spring Harbor Press, N.Y., (3^{rd} ed., 2000); Brent et al., Current Protocols in Molecular Biology, John Wiley & Sons, Inc. (ringbou ed., 2003); Davis et al., Basic Methods in Molecular Biology, Elsevier Science Publishing, Inc., New York, USA (1986); or Methods in Enzymology: Guide to Molecular Cloning Techniques Vol. 152, S. L. Berger and A. R. Kimmerl Eds., Academic Press Inc., San Diego, USA (1987); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998). The following sections provide additional guidance for practicing the compositions and methods of the present invention.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). Further clarifications of some of these terms as they apply specifically to this invention are provided herein.

As used herein, the singular forms “a,” “an,” and “the,” refer to both the singular as well as plural, unless the context clearly indicates otherwise. For example, “an Env-derived trimer” can refer to both single or plural Env-derived trimer molecules, and can be considered equivalent to the phrase “at least one Env-derived trimer.”

As used herein, the terms “antigen” or “immunogen” are used interchangeably to refer to a substance, typically a protein, which is capable of inducing an immune response in a subject. The term also refers to proteins that are immunologically active in the sense that once administered to a subject (either directly or by administering to the subject a nucleotide sequence or vector that encodes the protein) is able to evoke an immune response of the humoral and/or cellular type directed against that protein. Unless otherwise noted, the term “vaccine immunogen” is used interchangeably with “protein antigen” or “immunogen polypeptide”.

The term “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For polypeptide sequences, “conservatively modified variants” refer to a variant which has conservative amino acid substitutions, amino acid residues replaced with other amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Epitope refers to an antigenic determinant. These are particular chemical groups or peptide sequences on a molecule that are antigenic, such that they elicit a specific immune response, for example, an epitope is the region of an antigen to which B and/or T cells respond. Epitopes can be formed both from contiguous amino acids or noncontiguous amino acids juxtaposed by tertiary folding of a protein.

Effective amount of a vaccine or other agent that is sufficient to generate a desired response, such as reduce or eliminate a sign or symptom of a condition or disease, such as AIDS. For instance, this can be the amount necessary to inhibit viral replication or to measurably alter outward symptoms of the viral infection, such as increase of T cell counts in the case of an HIV-1 infection. In general, this amount will be sufficient to measurably inhibit virus (for example, HIV) replication or infectivity. When administered to a subject, a dosage will generally be used that will achieve target tissue concentrations (for example, in lymphocytes) that has been shown to achieve in vitro inhibition of viral replication. In some embodiments, an “effective amount” is one that treats (including prophylaxis) one or more symptoms and/or underlying causes of any of a disorder or disease, for example to treat HIV-1 infection. In some embodiments, an effective amount is a therapeutically effective amount. In some embodiments, an effective amount is an amount that prevents one or more signs or symptoms of a particular disease or condition from developing, such as one or more signs or symptoms associated with AIDS.

Unless otherwise noted, the term “responder frequency of a vaccine” (or just “responder frequency”) is used herein interchangeably with “frequency of vaccine responders”. They refer to the frequency of vaccinated subjects that can generate a sufficient level of neutralizing response within a test group.

As used herein, a fusion protein is a recombinant protein containing amino acid sequence from at least two unrelated proteins that have been joined together, via a peptide bond, to make a single protein. The unrelated amino acid sequences can be joined directly to each other or they can be joined using a linker sequence. As used herein, proteins are unrelated, if their amino acid sequences are not normally found joined together via a peptide bond in their natural environment(s) (e.g., inside a cell). For example, the amino acid sequences of bacterial enzymes such as B. stearothermophilus dihydrolipoyl acyltransferase (E2p) and the amino acid sequences of HIV-1 gp120 or gp41 glycoproteins are not normally found joined together via a peptide bond.

A heptad repeat (HR) refers to a structural motif that consists of a repeating pattern of seven amino acids: ab c d e f g H P H C P C. where H represents hydrophobic residues, C represents, typically, charged residues, and P represents polar (and, therefore, hydrophilic) residues.

HIV-1 envelope protein (Env) is initially synthesized as a longer precursor protein of 845-870 amino acids in size, designated gp160. gp160 forms a homotrimer and undergoes glycosylation within the Golgi apparatus. In vivo, gp160 glycoprotein is endo-proteolytically processed to the mature envelope glycoproteins gp120 and gp41, which are noncovalently associated with each other in a complex on the surface of the virus. The gp120 surface protein contains the high affinity binding site for human CD4, the primary receptor for HIV, as well as domains that interact with fusion coreceptors, such as the chemokine receptors CCR5 and CXCR4. The gp41 protein spans the viral membrane and contains at its amino-terminus a sequence of amino acids important for the fusion of viral and cellular membranes. The native, fusion-competent form of the HIV-1 envelope glycoprotein complex is a trimeric structure composed of three gp120 and three gp41 subunits. The receptor-binding (CD4 and co-receptor) sites are located in the gp120 moieties, whereas the fusion peptides are located in the gp41 components. Exemplary sequence of wildtype gp160 polypeptides are shown in GenBank, e.g., under accession numbers AAB05604 and AAD12142.

gp140 refers to an oligomeric form of HIV envelope protein, which contains all of gp120 and the entire gp41 ectodomain. As used herein, an HIV-1 gp140 trimer immunogen typically contains a gp140 domain and a modified or redesigned ectodomain of gp140 (gp41_{ECTO}).

gp120 is an envelope protein of the Human Immunodeficiency Virus (HIV). gp120 contains most of the external, surface-exposed, domains of the HIV envelope glycoprotein complex, and it is gp120 which binds both to cellular CD4 receptors and to cellular chemokine receptors (such as CCR5). The mature gp120 wildtype polypeptides have about 500 amino acids in the primary sequence. Gp120 is heavily N-glycosylated giving rise to an apparent molecular weight of 120 kD. The polypeptide is comprised of five conserved regions (C1-05) and five regions of high variability (V1-V5). In its tertiary structure, the gp120 glycoprotein is comprised of three major structural domains (the outer domain, the inner domain, and the bridging sheet) plus the variable loops. See, e.g., Wyatt et al., Nature 393, 705-711, 1998; and Kwong et al., Nature 393, 649-59, 1998. The inner domain is believed to interact with the gp41 envelope glycoprotein, while the outer domain is exposed on the assembled envelope glycoprotein trimer.

Variable region 1 and Variable Region 2 (V1/V2 domain) of gp120 are comprised of about 50-90 residues which contain two of the most variable portions of HIV-1 (the V1 loop and the V2 loop), and one in ten residues of the V1/V2 domain are N-glycosylated.

gp41 is a proteolytic product of the precursor HIV envelope protein. It contains an N-terminal fusion peptide (FP), a transmembrane domain, as well as an ectodomain that links the fusion peptide and a transmembrane domain. gp41 remains in a trimeric configuration and interacts with gp120 in a non-covalent manner. The amino acid sequence of an exemplary gp41 is set forth in GenBank, under Accession No. CAD20975.

BG505 SOSIP.664 gp140 is an HIV-1 Env immunogen developed with the gp140 trimer from clade-A strain BG505. It contains a covalent linkage between the cleaved gp120 and gp41_{ECTO} with an engineered disulfide bond (termed SOS). In addition, it has an I559P mutation (termed IP) to destabilize the gp41 post-fusion conformation and also a truncation of the membrane-proximal external region (MPER) at residue 664 to improve solubility. This HIV-1 immunogen has an outstanding antigenic profile and excellent structural mimicry of the native spike. Using the SOSIP trimer as a sorting probe, new bNAbs have been identified and characterized. The SOSIP design has also been extended to other HIV-1 strains and permitted the incorporation of additional stabilizing mutations. Recently, immunogenicity of SOSIP trimers in rabbits and nonhuman primates was reported, paving the way for human vaccine trials.

Unless otherwise noted, amino acid numbering in the HIV-1 Env or gp140 protein is based on the HXB2 system. HXB2 numbering system is a reference numbering system for HIV protein and nucleic acid sequences, using HIV-1 HXB2 strain sequences as a reference for all other HIV strain sequences. The person of ordinary skill in the art is familiar with the HXB2 numbering system, and this system is set forth in “Numbering Positions in HIV Relative to HXB2CG,” Bette Korber et al., Human Retroviruses and AIDS 1998: A Compilation and Analysis of Nucleic Acid and Amino Acid Sequences. Korber B, Kuiken C L, Foley B, Hahn B, McCutchan F, Mellors J W, and Sodroski J, Eds. Theoretical Biology and Biophysics Group, Los Alamos National Laboratory, Los Alamos, N. Mex.

Immunogen is a protein or a portion thereof that is capable of inducing an immune response in a mammal, such as a mammal infected or at risk of infection with a pathogen. Administration of an immunogen can lead to protective immunity and/or proactive immunity against a pathogen of interest.

Immune response refers to a response of a cell of the immune system, such as a B cell, T cell, or monocyte, to a stimulus. In some embodiment, the response is specific for a particular antigen (an “antigen-specific response”). In some embodiments, an immune response is a T cell response, such as a CD4+ response or a CD8+ response. In some other embodiments, the response is a B cell response, and results in the production of specific antibodies.

Immunogenic composition refers to a composition comprising an immunogenic polypeptide that induces a measurable CTL response against virus expressing the immunogenic polypeptide, or induces a measurable B cell response (such as production of antibodies) against the immunogenic polypeptide.

Sequence identity or similarity between two or more nucleic acid sequences, or two or more amino acid sequences, is expressed in terms of the identity or similarity between the sequences. Sequence identity can be measured in terms of percentage identity; the higher the percentage, the more identical the sequences are. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

Homologs or orthologs of nucleic acid or amino acid sequences possess a relatively high degree of sequence identity/similarity when aligned using standard methods. Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

N-linked glycosylation is the attachment of an oligosaccharide, a carbohydrate consisting of several sugar molecules, sometimes also referred to as glycan, to a nitrogen atom (the amide nitrogen) of an asparagine (Asn) residue of a protein. This type of linkage is important for both the structure and function of many eukaryotic proteins. The N-linked glycosylation process occurs in eukaryotes and widely in archaea, but very rarely in bacteria. The nature of N-linked glycans attached to a glycoprotein is determined by the protein and the cell in which it is expressed. It also varies across species. Different species synthesize different types of N-linked glycan.

The term “subject” refers to any animal classified as a mammal, e.g., human and non-human mammals. Examples of non-human animals include dogs, cats, cattle, horses, sheep, pigs, goats, rabbits, and etc. Unless otherwise noted, the terms “patient” or “subject” are used herein interchangeably. Preferably, the subject is human.

The term “treating” or “alleviating” includes the administration of compounds or agents to a subject to prevent or delay the onset of the symptoms, complications, or biochemical indicia of a disease (e.g., an HIV infection), alleviating the symptoms or arresting or inhibiting further development of the disease, condition, or disorder. Subjects in need of treatment include those already suffering from the disease or disorder as well as those being at risk of developing the disorder. Treatment may be prophylactic (to prevent or delay the onset of the disease, or to prevent the manifestation of clinical or subclinical symptoms thereof) or therapeutic suppression or alleviation of symptoms after the manifestation of the disease.

As used herein, uncleaved pre-fusion-optimized (UFO) trimers refer to HIV-1 gp140 trimeric proteins that are formed with gp120 protein and a redesigned gp41_{ECTO} domain, which results in more stabilized HIV-1 gp140 trimers. The redesigned gp41_{ECTO} domain is based on the prototype HIV-1 strain BG505 (and the prototype gp140 trimer BG505 SOSIP.664 gp140) and contains one or more modifications relative to the wildtype BG505 gp41_{ECTO} sequence. These modifications include (1) replacement of the 21 residue N-terminus of HR1 (residues 548-568) with a shorter loop sequence to stabilize the pre-fusion gp140 structure and (2) replacement of the furin cleavage site between gp120 and gp41 (residues 508-511) with a flexible linker sequence such a tandem repeat of a GGGGS (SEQ ID NO:3) motif. In some embodiments, the UFO trimer can additionally contain an engineered disulfide bond between gp120 and gp41 and/or a stabilizing mutation in gp41. For example, UFO trimers based on HIV-1 strain BG505 can contain an engineered disulfide bond between residues A501C and T605C. Detailed description of UFO trimers is provided in, e.g., Kong et al., Nat. Comm. 7:12040, 2016. In addition to UFO trimers based on the BG505 strain sequence, the engineered gp41_{ECTO} domain can be used to pair with a gp120 polypeptide from many different HIV-1 strains or subtypes to form “chimeric” gp140 trimers. Such chimeric trimers are termed “UFO-BG” or “UFO²-BG” as exemplified herein. Detailed description of UFO-BG and UFO²-BG trimers is provided in, e.g., He et al., Sci Adv. 4(11):eaau6769, 2018; and WO 2019/089817.

Vaccine refers to a pharmaceutical composition that elicits a prophylactic or therapeutic immune response in a subject. In some cases, the immune response is a protective immune response. Typically, a vaccine elicits an antigen-specific immune response to an antigen of a pathogen, for example a viral pathogen, or to a cellular constituent correlated with a pathological condition. A vaccine may include a polynucleotide (such as a nucleic acid encoding a disclosed antigen), a peptide or polypeptide (such as a disclosed antigen), a virus, a cell or one or more cellular constituents. In some embodiments of the invention, vaccines or vaccine immunogens or vaccine compositions are expressed from fusion constructs and self-assemble into nanoparticles displaying an immunogen polypeptide or protein on the surface.

Virus-like particle (VLP) refers to a non-replicating, viral shell, derived from any of several viruses. VLPs are generally composed of one or more viral proteins, such as, but not limited to, those proteins referred to as capsid, coat, shell, surface and/or envelope proteins, or particle-forming polypeptides derived from these proteins. VLPs can form spontaneously upon recombinant expression of the protein in an appropriate expression system. Methods for producing particular VLPs are known in the art. The presence of VLPs following recombinant expression of viral proteins can be detected using conventional techniques known in the art, such as by electron microscopy, biophysical characterization, and the like. See, for example, Baker et al. (1991) Biophys. J. 60:1445-1456; and Hagensee et al. (1994) J. Virol. 68:4503-4505. For example, VLPs can be isolated by density gradient centrifugation and/or identified by characteristic density banding. Alternatively, cryoelectron microscopy can be performed on vitrified aqueous samples of the VLP preparation in question, and images recorded under appropriate exposure conditions.

A self-assembling nanoparticle refers to a ball-shape protein shell with a diameter of tens of nanometers and well-defined surface geometry that is formed by identical copies of a non-viral protein capable of automatically assembling into a nanoparticle with a similar appearance to VLPs. Known examples include ferritin (FR), which is conserved across species and forms a 24-mer, as well as B. stearothermophilus dihydrolipoyl acyltransferase (E2p), Aquifex aeolicus lumazine synthase (LS), and Thermotoga maritima encapsulin, which all form 60-mers. Self-assembling nanoparticles can form spontaneously upon recombinant expression of the protein in an appropriate expression system. Methods for nanoparticle production, detection, and characterization can be conducted using the same techniques developed for VLPs.

### III. Nanoparticle Scaffolded Vaccines for Glycan Trimming

In one aspect, the invention provides methods for improving activities (e.g., immunogenicity and responder frequency) of nanoparticle scaffolded vaccines. The vaccines can be derived from any viral immunogenic proteins with a glycan shield. Specific examples of such immunogenic proteins are described herein. Also encompassed by the invention are vaccines treated with methods of the invention, which contain trimmed or shortened N-linked glycan chains. In some methods, the employed vaccines are HIV-1 nanoparticle vaccines. In some other embodiments, the employed vaccines are based on immunogenic proteins from other viruses as described below. As demonstrated herein, the HIV-1 gp140 based vaccines with trimmed or shortened glycan chains have enhanced immunogenicity and neutralizing antibody inducing activities. In various embodiments, HIV-1 nanoparticle vaccines with trimmed or shortened glycan chains can be produced by either post-expression enzymatic treatment of vaccine proteins or expression of the vaccine constructs in an expression system that lacks normal glycosylation function for human proteins. In some embodiments, the HIV-1 vaccines for glycan trimming or shortening in the practice of the present invention contain an HIV-1 Env derived trimer protein that is displayed on a self-assembling nanoparticle scaffold.

Any Env-derived HIV-1 trimer proteins can be used in the starting HIV-1 nanoparticle vaccines for glycan trimming or shortening. Various examples of such proteins are described below. The Env-derived trimer protein can be obtained from various HIV-1 strains. In some embodiments, the nanoparticles present a native trimeric form of HIV-1 Env based glycoproteins or domains, e.g., gp140, gp120 or V1V2 domains. In some embodiments, the employed HIV-1 Env-derived trimer protein is an uncleaved prefusion-optimized (UFO) gp140 trimer. In some embodiments, the Env-derived trimer is from HIV-1 strain BG505, e.g., the BG505.SOSIP.664 gp140 trimer. In some preferred embodiments, the starting nanoparticles present a modified gp140 trimer immunogen, e.g., a HR1-modified gp140 trimer (“UFO trimer”) described in Kong et al., Nat. Comm. 7, 12040, 2016. The amino acid sequence of subunit of this HR1-modified gp140 trimer protein is shown in SEQ ID NO:4. In some embodiments, the HIV-1 trimeric immunogen displayed in the starting nanoparticle vaccine in the invention can be a UFO²-BG trimer. UFO²-BG trimers are chimeric gp140 trimers containing (1) the BG505 gp41 domain with a redesigned HR1 N-terminal bend and a cleavage-site linker (as described in Kong et al., Nat. Comm. 7, 12040, 2016) and (2) the gp120 protein from one of other diverse HIV-1 strains or subtypes. In addition to the redesigned gp41_{ECTO} domain from the BG505 strain, the gp41 domain in the chimeric gp140 trimers suitable for the invention can also be a consensus gp41_{ECTO} domain derived from the HIV-1 sequence database. Many other HIV-1 Env derived trimer sequences known in the art can also be used in the starting nanoparticle vaccines of the invention. See, e.g., WO2017/192434, WO2019/089817, and WO2019/241483. Also can be used in constructing the starting HIV-1 nanoparticle vaccines in the practice of the invention are conservatively modified variants of the various HIV-1 trimer proteins described herein, or variants with substantially identical sequences thereof.

The displaying scaffold used in the starting HIV-1 vaccines for practicing the methods of the invention can be any self-assembling nanoparticle sequence. In general, the nanoparticles employed in the invention need to be formed by multiple copies of a single subunit. In some preferred embodiments, the employed self-assembling nanoparticles are derived from ferritin (FR), E2p and 13-01 as exemplified herein. Examples of these scaffold sequences are shown in SEQ ID NOs:1 and 2. Other suitable scaffold sequences are also known in the art. See, e.g., WO2017/192434, WO2019/089817, and WO2019/241483. Two starting HIV-1 nanoparticle vaccines, which respectively utilize an E2p derived scaffold sequence (SEQ ID NO:1) and an 13-01 variant scaffold sequence (SEQ ID NO:2), are exemplified herein. In various embodiments, the starting HIV-1 nanoparticle vaccines for practicing methods of the invention can employ any of these known nanoparticle scaffolds, as well as their conservatively modified variants or variants with substantially identical (e.g., at least 90%, 95% or 99% identical) sequences.

In addition to the HIV-1 Env trimer and the displaying scaffold, the starting nanoparticle vaccines can also contain other structural components. In some embodiments, a locking domain is fused at the C-terminus to the nanoparticle sequence. The locking domain functions to stabilize the nanoparticles from the inside in displaying the immunogen protein or polypeptide (e.g., Env-derived HIV-1 trimer protein). In general, the locking domain can be any protein capable of forming a dimer. Typically, the locking domain is covalently fused to the nanoparticle subunit to which the immunogen polypeptide (e.g., subunit of an HIV-1 Env derived trimer protein) is linked. Many proteins known in the art can be employed as the locking domain in the practice of the invention. See, e.g., WO2019/241483. Two specific locking domain sequences (SEQ ID NO:5 and SEQ ID NO:6) are present in the starting HIV-1 SApNP vaccines exemplified herein.

In some embodiments, the starting HIV-1 SApNP vaccines for glycan trimming or shortening can additionally contain a T-cell epitope to promote robust T-cell responses and to steer B cell development towards bNAbs. The T-cell epitope can be located at any position in relation to the other structural components so long as it does not impact presentation of the immunogen polypeptides on the nanoparticle surface. In some preferred embodiments, the T-cell epitope is located at the C-terminus of the nanoparticle subunit, e.g., by fusing the N-terminus of the T-cell epitope to the C-terminus of the locking domain subunit sequence, as exemplified in the nanoparticle vaccines described in the Examples herein. Any T helper epitope known in the art can be used in constructing the starting vaccine compositions in the practice of the invention. See, e.g., Alexander et al., Immunity 1, 751-761, 1994; Ahlers et al., J. Clin. Invest. 108:1677-1685, 2001; Fraser et al., Vaccine 32, 2896-2903, 2014; De Groot et al., Immunol. Cell Biol. 8:255-269, 2002; and Smahel et al., Gene Ther. 21: 225-232, 2014. In some preferred embodiments, the employed T-helper epitope is the universal pan-reactive T-cell epitope peptide, AKFVAAWTLKAAA (SEQ ID NO:7).

In various embodiments, nanparticles displaying any of the immunogen polypeptides or proteins described herein (e.g., HIV-1 Env-derived trimer immunogens) can be constructed by fusing the immunogen polypeptide or subunit of multimeric immunogen protein (e.g., a trimer immunogen) to the subunit of the nanoparticle (e.g., E2p or I3-01 subunit) and the locking domain, as well as the other optional or alternative components as needed or helpful (e.g., linker moieties or foldon). To construct the nanoparticle displayed fusion vaccine immunogens of the invention, one or more linker motifs or moieties may be employed to facilitate connection and maintain structural integrity of the different components. Thus, in some embodiments, a linker motif can be employed to connect the C-terminus of the immunogen polypeptide (e.g., HIV-1 trimer protein subunit) to the N-terminus of the nanoparticle subunit. Additionally or alternatively, a second linker motif can be used to link the C-terminus of the nanoparticle subunit (or the C-terminus of the immunogen polypeptide) to the N-terminus of the locking domain. In some other embodiments, a third linker motif may be employed to connect the T-cell epitope, e.g., linking the C-terminus of the locking domain to the N-terminus of the T-cell epitope, or linking the C-terminus of the T-cell epitope to the N-terminus of the locking domain. In some embodiments, linkers can also be used to insert a neck domain or a foldon domain into the nanoparticle vaccine constructs. Typically, the linker motifs contain short peptide sequences. In various embodiments, the linkers or linker motifs can be any flexible peptides that connect two protein domains without interfering with their functions. For example, any of these linkers used in the constructs can be GC-rich peptides with a sequence of (GₐS_{b}) n, wherein a is an integer of about 1-5, b is an integer of about 0-2, and n is an integer of about 1-5. In some other embodiments, a T-cell epitope can be used as a linker or part of a linker between the C-terminus of the immunogen polypeptide and the N-terminus of the nanoparticle subunit.

The starting HIV-1 vaccine compositions in the practice of the invention can be constructed recombinantly in accordance with methods that have been described in the art, e.g., WO2019/089817; WO2019/241483; He et al., Nat. Comm. 7, 12041, 2016; Kong et al., Nat. Comm. 7, 12040, 2016; and He et al., Sci Adv. 4(11):eaau6769, 2018. As exemplification, two specific HIV-1 nanoparticle vaccine constructs are described herein. The first construct expresses a fusion polypeptide that contains from the N-terminus to the C-terminus: HIV-1 UFO BG505.SOSIP.664 gp140 subunit sequence, E2p nanoparticle subunit sequence (e.g., SEQ ID NO:1), a linker motif (GₐS_{b})ₙ noted above (e.g., (GGGGS)₂ (SEQ ID NO:8)), a locking domain as shown in SEQ ID NO:5 (LD4), and a T-cell epitope (e.g., the PADRE epitope shown in SEQ ID NO:7). Optionally, the immunogen polypeptide (e.g., gp140 subunit for HIV-1 vaccine) can be connected to the nanoparticle subunit (e.g., E2p) via a linker sequence, e.g., GGGGS (SEQ ID NO:3) or (GGGGS)₂ (e.g., SEQ ID NO:8). The second construct expresses a fusion polypeptide that contains from the N-terminus to the C-terminus: HIV-1 UFO BG505.SOSIP.664 gp140, a linker sequence (GGGGS)₂ (SEQ ID NO:8), I3-01 nanoparticle subunit sequence (SEQ ID NO:2), a second linker (GₐS_{b})ₙ noted above (e.g., GGGGS (SEQ ID NO:3)), a locking domain as shown in SEQ ID NO:6 (LD7), and a T-cell epitope (e.g., the epitope as shown in SEQ ID NO:7). Optionally, a dipeptide linker, GS, can be inserted between the locking domain and the T-cell epitope in any of these vaccine constructs. The antigeniciy and structural integrity of the vaccine immunogens (e.g., HIV-1 nanoparticle immunogens) can be readily analyzed via standard assays, e.g., antibody binding assays and negative-stain electron microscopy (EM). As exemplified herein, the fusion molecules can all self-assemble into nanoparticles that display immunogenic epitopes of the Env-derived trimer (e.g., gp140).

### IV. Non-Scaffolded Viral Immunogenic Proteins for Glycan Trimming

The same strategy of glycan trimming described above for nanoparticle scaffolded viral proteins can be equally applied to non-scaffolded vaccines. These include any HIV-1 Env derived trimmer immunogens and also vaccines derived from immunogenic proteins of other viruses with glycan shield. In some embodiments, the invention provides methods for enhancing immunogenicity and responder frequency of non-scaffolded HIV-1 Env trimer proteins by glycan trimming. HIV-1 Env is a heterodimer of a transmembrane glycoprotein (gp41) and a surface glycoprotein (gp120). These dimers are organized as trimers on the surface of the viral membrane. In some preferred embodiments, the employed HIV-1 Env derived immunogen is a gp140 trimer, not a full length Env gp160 protein or just a gp120 protein alone. Instead, these engineered HIV-1 Env gp140 trimer immunogens typically contain a gp120-derived polypeptide and a redesigned gp41-derived polypeptide with a redesigned N-terminus (residues 548-568) of the heptad region 1 (HR1) in gp41. The gp120-derived polypeptide and the gp41-derived polypeptide can be associated non-covalently as in the natural HIV-1 gp140 protein or covalently linked via a linker sequence described herein. The gp120-derived polypeptide does not need to contain the full-length sequence of a wildtype gp120 glycoprotein. Thus, in various embodiments, the gp120-derived polypeptide can be, e.g., the natural gp120 protein, the V1V2 domains of the gp120 glycoprotein, the gp120 core (i.e., the inner domain and the outer domain), and just the outer domain of gp120 core. In some embodiments, the employed gp120-derived polypeptide encompasses its gp41-interactive region and the antigenic epitopes (e.g., the outer domain).

In some embodiments, the gp120- and gp41-derived polypeptides in the HIV-1 trimer immunogens are derived from the same HIV-1 strain or subtype. In some other embodiments, the gp120 and gp120 polypeptides can be derived from different HIV-1 strains or subtypes. For example, a modified gp41 from strain BG505 or a universal gp41 domain derived from the HIV-1 sequence database can be combined with gp120 from various other HIV-strains or subtypes to form different chimeric HIV-1 trimer immunogens. In general, the gp41-derived polypeptide in the HIV-1 trimer immunogens typically harbors the HR1 region of the native gp41 protein excerpt for some modifications at the N-terminus. In some embodiments, the gp41-derived polypeptide is a soluble polypeptide that has the transmembrane region truncated, e.g., a polypeptide containing the ectodomain (gp41_{ECTO}) or a polypeptide containing the fusion peptide and the ectodomain. In some preferred embodiments, the employed HIV-1 trimer immunogens for glycan trimming are HIV-1 UFO trimers as noted above in nanoparticle scaffolded vaccines. In these HIV-1 Env trimmer immunogens, the 21 residue N-terminus of HR1 (residues 548-568) of the gp41-derived polypeptide is replaced with a shorter loop sequence to stabilize the pre-fusion gp140 structure. For example, some HIV-1 UFO trimer immunogens suitable for methods of the invention have the 21 aa HR1 motif in gp41_{ECTO} replaced with a 8 aa loop sequence NPDWLPDM (SEQ ID NO:9). Detailed guidance for obtaining suitable replacing loop sequences in the UFO trimmers are described in, e.g., Kong et al., Nat. Comm. 7:12040, 2016; and WO 2017/192434.

Other than the HR1 N-terminus modification, some HIV-1 trimer immunogens suitable for methods of the invention also have the protease cleavage site between gp120 and gp41 replaced with a linker sequence to create non-cleavable gp140 protein. In different embodiments, the replaced cleavage site can be a 4-residue cleavage site (i.e., residues 508-511) or a longer cleavage site-containing region (e.g., residues 501-518). In different embodiments, the replacing linkers can contain different amino acid residues of varying length. As exemplified in some HIV-1 UFO trimers, the cleavage site can be replaced with a linker of (G₄S)₂ (SEQ ID NO:8). In some of these embodiments, the replaced cleavage site contains residues 501-518. In some preferred embodiments, the employed HIV-1 trimer immunogens contain a combination of the cleavage site linker sequence and the redesigned HR1 N-terminus noted above.

The employed HIV-1 trimmer immunogens can contain additional modifications relative to the wildtype sequences. In some embodiments, the association between gp120 and gp41 can be stabilized by the introduction of a correctly positioned intermolecular disulfide bond to make a soluble form of Env. The gp120-gp41 interactions in such a gp140 protein can also be stabilized by deleting the first and second variable (V1 and V2) loops of the gp120 polypeptide and by introducing amino acid substitutions into the N-terminal heptad repeat region around position 559 of gp41 (e.g., I559P mutation). These structural modifications are exemplified in the SOSIP HIV-1 gp140 trimer. Further modifications to the HIV-1 gp140 may include modified glycan site at residue 332 (T332N). In some other embodiments, the employed HIV-1 trimmer immunogen that harbors the above-noted redesigned HR1 N-terminus also has other mutations or alterations introduced at the cleavage site, e.g., replacing REKR (SEQ ID NO:10) with RRRRRR (SEQ ID NO:11). In various embodiments, the C terminus of the modified HIV-1 gp140 protein trimmer can be truncated to either residue 664 or 681 (according to HXB2 nomenclature), resulting in the two gp140 versions like “BG505 SOSIP.gp140.664” and “BG505 SOSIP.gp140.681” which are well known in the art. HIV-1 vaccines based on BG505 SOSIP.gp140.664 are employed in some of the studies exemplified herein.

While the methods of the invention are exemplified with vaccines based on the BG505 strain, they can also employ HIV-1 trimmer immunogens derived from various other HIV-1 clades or strains. HIV-I can be classified into four groups: the “major” group M, the “outlier” group 0, group N, and group P. Within group M, there are several genetically distinct clades (or subtypes) of HIV-I. The employed HIV-1 trimmer immunogens in the present invention can be derived from any subtype of HIV, such as groups M, N, O, or P or Glade A, B, C, D, F, G, H, J or K and the like. Sequences encoding HIV-1 Env glycoproteins and methods for the manipulation and insertion of such nucleic acid sequences into vectors, are known (see, e.g., HIV Sequence Compendium, Division of AIDS, National Institute of Allergy and Infectious Diseases (2003); HIV Sequence Database; Sambrook et al., Molecular Cloning: A Laboratory Manual, Cold Spring Harbor Press, N.Y., (3^{rd} ed., 2000); and Brent et al., Current Protocols in Molecular Biology, John Wiley & Sons, Inc. (ringbou ed., 2003).

In addition to HIV-1 Env trimmer based vaccines, vaccines derived from immunogenic proteins of any other viruses that contain a glycan shield are also suitable for the methods of the invention. For example, vaccines developed with envelope proteins or spike proteins of a number of viruses with a dense glycan shield can be readily employed in the practice of the invention. These immunogenic proteins include, e.g., proteins from Lassa viruses (e.g., envelope glycoprotein complex, GPC), hepatitis C virus (e.g., E2 envelope glycoprotein), influenza virus (e.g., surface glycoprotein hemagglutinin), and SARS-CoV-2 (e.g., spike protein). See, e.g., Lavie et al., Front. Immunol. 9: 910, 2018; LeBlanc et al., Pathogens 10: 685, 2021; Sommerstein et al., PLoS Pathog. 11: e1005276, 2015; Watanabe et al., Proc. Natl. Acad. Sci. USA 115: 7320-25, 2018; Wanzeck et al., Am. J. Respir. Crit. Care Med. 183: 767-73, 2011; Altman et al., mBio 10: e00204-19, 2019; Casalino et al., ACS Cent. Sci. 6: 1722-34, 2020; and Zhao et al., Front. Mol. Biosci. 8: 629873, 2021.

V. Vaccines with Trimmed or Shortened Glycan Chain

The invention provides methods for generating vaccines with trimmed or shortened N-linked glycan chains on viral immunogenic proteins, as well as related vaccine compositions thus produced. As described above, both nanoparticle scaffolded and non-scaffolded viral immunogenic proteins are included in the invention. In some embodiments, the methods involve post-expression trimming of the glycan chains on the vaccine proteins. As demonstrated herein, the glycan trimming step can significantly increase the vaccine immunogenicity and induce potent neutralizing antibody responses in more subjects during vaccination. In some embodiments, glycan trimming of the vaccines (e.g., soluble HIV-1 UFO trimers or nanoparticle vaccines) is achieved via enzymatic digestion. In these embodiments, the expressed and optionally purified vaccines are subject to in vitro glycan-trimming with an enzyme. Any enzyme that is capable of digesting the N-linked glycan chain on the viral proteins (e.g., HIV-1 Env trimer proteins) can be employed in the practice of the invention. In general, glycan chains can be trimmed with glycosidases which catalyze the hydrolysis of glycosidic bonds to remove sugars from proteins. These enzymes are critical for glycan processing in the ER and Golgi, and each enzyme shows specificity for removing a particular sugar (e.g., mannosidase).

In some preferred embodiments, the enzyme to be used is endoglycosidase H (Endo-H), which cleaves within the chitobiose core of high mannose and some hybrid oligosaccharides from N-linked glycoproteins. Endo-H is a 29 kD protein isolated from Streptomyces plicatus or Streptomyces griseus. Structure and activities of this enzyme are well known in the art. See, e.g., Robbins et al., J. Biol. Chem. 259: 7577-83, 1984; and Trimble et al., Analy. Chem. 141: 515-22, 1984. It cleaves the bond in the diacetylchitobiose core of the oligosaccharide between two N-acetylglucosamine (GlcNAc) subunits directly proximal to the asparagine residue, generating a truncated sugar molecule with one N-acetylglucosamine residue remaining on the asparagine. It deglycosylates mannose glycoproteins, but the extent and rate of the deglycosylation depends to a high degree on the nature of the glycoproteins. The deglycosylation rate can be increased by denaturation of the glycoproteins (e.g., by carboxymethylation, sulfitolysis or by heating in the presence of sodium dodecyl sulfate). The addition of 2-mercaptoethanol can significantly increase the enzyme's activity against glycoproteins containing inter- or intra-molecular disulfide bridges, unlike detergents like Triton X-100, n-Octylglucoside, or zwitterionic detergents. In the practice of the invention, the Endo-H enzyme can be readily obtained from commercial vendors, e.g., New England BioLabs. Glycan trimming of viral immunogenic proteins with the enzyme can be performed according to the instructions of the enzyme manufacturer (e.g., New England BioLabs). Optionally, some minor modifications of the standard protocols can be implemented to ensure desired digestion of the N-linked glycan chain on the viral immunogen polypeptides (e.g., HIV-1 UFO trimer either in soluble form or displayed on a nanoparticle scaffold).

In various embodiments, the N-linked glycan chains on the viral immunogenic proteins (e.g., a modified HIV-1 gp140 protein or UFO trimer) are trimmed or shortened to different degrees. In some embodiments, the entire glycan chains are trimmed. In some of these embodiments, the Endo-H treatment can be conducted at room temperature (e.g., at 25° C.) using purified vaccine material (e.g., HIV-1 SApNP vaccine or un-scaffolded UFO trimer) without denaturing. In some of these embodiments, the maximum enzyme/vaccine protein ratio suggested by the enzyme manufacturer is used to ensure the most complete enzymatic digestion of N-linked glycans on the immunogen protein surface. For example, as exemplified herein with both non-scaffolded HIV-1 BG505 UFO trimer vaccine and Ep2 or 13-01 displayed trimer nanoparticle vaccine, 12,500 units or 25 μl of the enzyme (New England BioLabs) can be used to treat 100 μg of the purified vaccine proteins. In some embodiments, a less than complete trimming of the glycan chain is desired. For example, the trimming can entail shortening of the glycan chain by about 50%, 55%, 60%, 65%, 70%, 75%, 80%, 85%, 90%, or 95% of its length. In some of these embodiments, a lesser enzyme/protein ratio may be used to achieve a desired degree of glycan trimming. For example, for 100 μg of the HIV-1 vaccine material (e.g., SApNP or non-scaffolded UFO trimer), 10,000 units (20 μl), 7,500 units (15 μl), 5,000 units (10 μl), 2,500 units (5 μl), 1,250 units (2.5 μl) or even less of the exemplified Endo-H enzyme may be used. In still some other embodiments, a desired degree of glycan trimming can be achieved by adjusting the length of period for the enzymatic action. For example, to ensure proper and/or complete enzymatic processing, the reaction can last for at least 0.5 hr, 1 hr, 2 hrs, 3 hrs, 4 hrs, 5 hrs, 6, hrs, 7 hrs, 8 hrs, 9 hrs, 10 hrs, 15 hrs, 20 hrs or longer. In various embodiments, the enzymatic digestion can last from about 1 hr to about 8 hrs, from about 2 hrs to about 6 hrs, or from 3 hrs to about 4 hrs. In some embodiments, the amount of the enzyme used and the treatment time should enable a substantial reduction of the length of the glycan chains on the viral immunogenic protein. In various embodiments, the enzymatic treatment should lead to a shortening of the glycan chains by at least 50%, at least 55%, at least 60%, at least 65%, at least 70%, at least 75%, at least 80%, at least 85%, at least 90%, at least 95%, or at least 99% of their length. In these embodiments, substantially all glycan chains (e.g., at least 75%, at least 85%, at least 95% or all) present on the surface of the viral vaccine protein are shortened by the noted extent.

In addition to varying the condition of the enzymatic digestion (e.g., length of reaction time, amount of enzyme used and etc.), extent of glycan trimming can also be controlled by the use of different enzymes. Several classes of enzymes can be used alone, in combination, or in a particular sequential order to achieve different levels of glycan trimming by cleaving off residues from the tip of a glycan chain towards the Asp residue to which an N-linked glycan chain is attached. For example, Peptide/N-glycosidase F (PNGase F) cleaves between the innermost GlcNAc and Asparagine residues of high mannose, hybrid, and complex oligosaccharides, resulting in complete deglycosylation of an N-linked glycoproteins (100% trimming). PNGase F is sensitive to protein structure and may not cleave some or all of glycans unless the glycoprotein is fully denatured. Endoglycosidases (Endo) cleave between the N-acetylglucosamine residues within the glycan core and leave one N-acetylglucosamine residue on the Asparagine (approximately 70-90% trimming). These include, e.g., Endo-H noted above, as well as Endo-F1, F2 and F3. Endo-F1 and Endo-H cleave oligomannose and hybrid glycans, while Endo-F2 and Endo-F3 prefer to cleave complex glycans. Other enzymes, such as neuraminidase, β-galactosidase, N-acetylglucosaminidase, and fucosidases, can be used sequentially to trim a complex oligosaccharide to Man₃GlcNAc₂ (approximately 50-70% trimming). Therefore, glycan trimming to desirable levels can be achieved by using these enzymes in their functional conditions to cleave off residual groups from N-linked glycans. The ranges of optimal temperature, pH, and digestion time are well-documented for these enzymes.

Following glycan trimming, the processed vaccine material can be further subject to purification. Any standard or well-known protein purification methods may be employed in the practice of the invention. In some embodiments, the glycan trimmed vaccines can be purified via size-exclusion chromatography (SEC). For example, as exemplified herein, the Endo-H treated vaccines can be purified on a Superose 6 column. Additional routinely practiced protein purification techniques or other polishing steps can also be used in the purification of the glycan-trimmed vaccine compositions (e.g., scaffolded or non-scaffolded HIV-1 UFO trimer vaccine) described herein. These include, e.g., centrifugation, separation based on charge or hydrophobicity, affinity chromatography, and immunoaffinity chromatography.

Other than enzymatic trimming of post-expression vaccine protein materials, vaccine compositions (e.g., HIV-1 UFO trimer vaccine) with trimmed or shortened glycan chains can also be obtained by expressing the vaccine constructs in cell lines or expressing systems without normal post-translational N-glycosylation function. For example, most prokaryotic expression systems such as E. coli cannot carry out post-translational modifications. Non-human mammalian expression systems such as CHO or NS0 cells have the machinery required to add complex, human-type glycans. However, glycans produced in these systems can differ from glycans produced in humans, as they can be capped with both N-glycolylneuraminic acid (Neu5Gc) and N-acetylneuraminic acid (Neu5Ac), whereas human cells only produce glycoproteins containing N-acetylneuraminic acid.

In some embodiments, viral vaccines without normal glycan chains can be produced by the expressing the vaccine constructs in Sf9 insect cells. Proteins produced from these insect cells contain shorter Man₃GlcNAc₂ glycans. In some other embodiments, viral vaccines without normal glycan chains can be produced by the expressing the vaccine constructs in HEK293F GnTI-cells. These cells do not have N-acetylglucosaminyltransferase I (GnTI) activity, lack complex N-glycans, and produce Man₅₋₉GlcNAc₂ glycans.

LD4 (SEQ ID NO: 5):
FSEEQKKALDLAFYFDRRLTPEWRRYLSQRLGLNEEQIERWFRRKEQQIG
WSHPQFEK
LD7 (SEQ ID NO: 6):
SPAVDIGDRLDELEKALEALSAEDGHDDVGQRLESLLRRWNSRRAD
E2p subunit sequence (SEQ ID NO: 1)
AAAKPATTEGEFPETREKMSGIRRAIAKAMVHSKHTAPHVTLMDEADVTK
LVAHRKKFKAIAAEKGIKLTFLPYVVKALVSALREYPVLNTAIDDETEEI
IQKHYYNIGIAADTDRGLLVPVIKHADRKPIFALAQEINELAEKARDGKL
TPGEMKGASCTITNIGSAGGQWFTPVINHPEVAILGIGRIAEKPIVRDGE
IVAAPMLALSLSFDHRMIDGATAQKALNHIKRLLSDPELLLM
13-01 variant sequence (SEQ ID NO: 2)
MKMEELFKKHKIVAVLRANSVEEAKMKALAVFVGGVHLIEITFTVPDADT
VIKELSFLKELGAIIGAGTVTSVEQCRKAVESGAEFIVSPHLDEEISQFC
KEKGVFYMPGVMTPTELVKAMKLGHTILKLFPGEVVGPQFVKAMKGPFPN
VKFVPTGGVNLDNVCEWFKAGVLAVGVGSALVKGTIAEVAAKAAAFVEKI
RGCTE
Sequence of HR1-modified gp140 trimer
(SEQ ID NO: 4)
AENLWVTVYYGVPVWKDAETTLFCASDAKAYETEKHNVWATHACVPTDPN
PQEIHLENVTEEFNMWKNNMVEQMHTDIISLWDQSLKPCVKLTPLCVTLQ
CTNVTNNITDDMRGELKNCSFNMTTELRDKKQKVYSLFYRLDVVQINENQ
GNRSNNSNKEYRLINCNTSAITQACPKVSFEPIPIHYCAPAGFAILKCKD
KKFNGTGPCPSVSTVQCTHGIKPVVSTQLLLNGSLAEEEVMIRSENITNN
AKNILVQFNTPVQINCTRPNNNTRKSIRIGPGQAFYATGDIIGDIRQAHC
NVSKATWNETLGKVVKQLRKHFGNNTIIRFANSSGGDLEVTTHSFNCGGE
FFYCNTSGLFNSTWISNTSVQGSNSTGSNDSITLPCRIKQIINMWQRIGQ
AMYAPPIQGVIRCVSNITGLILTRDGGSTNSTTETFRPGGGDMRDNWRSE
LYKYKVVKIEPLGVAPTRCKRRVVGGGGGSGGGGSAVGIGAVFLGFLGAA
GSTMGAASMTLTVQARNLLSGNPDWLPDMTVWGIKQLQARVLAVERYLRD
QQLLGIWGCSGKLICCTNVPWNSSWSNRNLSEIWDNMTWLQWDKEISNYT
QIIYGLLEESQNQQEKNEQDLLALD

### VI. Pharmaceutical Compositions and Therapeutic Applications

The glycan trimmed viral vaccines (e.g., scaffolded or non-scaffolded HIV-1 vaccines) described herein and related pharmaceutical compositions can be used in prophylactic and therapeutic applications for treating or preventing HIV-1 infections. In the practice of the therapeutic methods of the invention, the subjects in need of prevention or treatment of HIV-1 infection is administered with an effective amount of the glycan trimmed vaccine or pharmaceutical composition. The administered pharmaceutical composition can be either a therapeutic formulation or a prophylactic formulation. Typically, the composition additionally includes one or more pharmaceutically acceptable vehicles and, optionally, other therapeutic ingredients (for example, antibiotics or antiviral drugs). Various pharmaceutically acceptable additives can also be used in the compositions.

For therapeutic applications, appropriate adjuvants can be additionally included in the glycan trimmed HIV-1 vaccine compositions of the invention. Examples of suitable adjuvants include, e.g., aluminum hydroxide, lecithin, Freund's adjuvant, MPL™ and IL-12. In some embodiments, the vaccine compositions can be formulated as a controlled-release or time-release formulation. This can be achieved in a composition that contains a slow release polymer or via a microencapsulated delivery system or bioadhesive gel. The various pharmaceutical compositions can be prepared in accordance with standard procedures well known in the art. See, e.g., Remington's Pharmaceutical Sciences, 19^{th} Ed., Mack Publishing Company, Easton, Pa., 1995; Sustained and Controlled Release Drug Delivery Systems, J. R. Robinson, ed., Marcel Dekker, Inc., New York, 1978); U.S. Pat. Nos. 4,652,441 and 4,917,893; 4,677,191 and 4,728,721; and 4,675,189.

In some therapeutic applications of the invention, a glycan trimmed vaccine (e.g., HIV-1 UFO trimer vaccine) composition can be administered to a subject to induce an immune response to the cognate virus (e.g., HIV-1), e.g., to induce production of broadly neutralizing antibodies to the virus. For subjects at risk of developing an HIV infection, a vaccine composition of the invention can be administered to provide prophylactic protection against viral infection. Depending on the specific subject and conditions, pharmaceutical compositions of the invention can be administered to subjects by a variety of administration modes known to the person of ordinary skill in the art, for example, intramuscular, subcutaneous, intravenous, intra-arterial, intra-articular, intraperitoneal, or parenteral routes. In general, the pharmaceutical composition is administered to a subject in need of such treatment for a time and under conditions sufficient to prevent, inhibit, and/or ameliorate an HIV-1 infection. The immunogenic composition is administered in an amount sufficient to induce an immune response against HIV-1. For therapeutic applications, the compositions should contain a therapeutically effective amount of the glycan trimmed immunogenic protein (e.g., HIV-1 UFO trimer immunogen) described herein. For prophylactic applications, the compositions should contain a prophylactically effective amount of the glycan trimmed viral immunogen described herein. The appropriate amount of the immunogen can be determined based on the specific disease or condition to be treated or prevented, severity, age of the subject, and other personal attributes of the specific subject (e.g., the general state of the subject's health and the robustness of the subject's immune system). Determination of effective dosages is additionally guided with animal model studies followed up by human clinical trials and is guided by administration protocols that significantly reduce the occurrence or severity of targeted disease symptoms or conditions in the subject.

For prophylactic applications, the immunogenic composition is provided in advance of any symptom, for example in advance of infection. The prophylactic administration of the immunogenic compositions serves to prevent or ameliorate any subsequent infection. Thus, in some embodiments, a subject to be treated is one who has, or is at risk for developing, an HIV-1 infection, for example because of exposure or the possibility of exposure to the HV-1 virus. Following administration of a therapeutically effective amount of the disclosed therapeutic compositions, the subject can be monitored for an infection, symptoms associated with an HIV-1 infection, or both.

For therapeutic applications, the immunogenic composition is provided at or after the onset of a symptom of disease or infection, for example after development of a symptom of infection, or after diagnosis of the infection. The immunogenic composition can thus be provided prior to the anticipated exposure to HIV virus so as to attenuate the anticipated severity, duration or extent of an infection and/or associated disease symptoms, after exposure or suspected exposure to the virus, or after the actual initiation of an infection.

The pharmaceutical composition of the invention can be combined with other agents known in the art for treating or preventing HIV-1 infections. For example, these known agents include, e.g., antibodies or other antiviral agents such as nucleoside reverse transcriptase inhibitors, such as abacavir, AZT, didanosine, emtricitabine, lamivudine, stavudine, tenofovir, zalcitabine, zidovudine, and the like, non-nucleoside reverse transcriptase inhibitors, such as delavirdine, efavirenz, nevirapine, protease inhibitors such as amprenavir, atazanavir, indinavir, lopinavir, nelfinavir, osamprenavir, ritonavir, saquinavir, tipranavir, and the like, and fusion protein inhibitors such as enfuvirtide and the like. Administration of the pharmaceutical composition and the known anti-HIV agents can be either concurrently or sequentially.

The glycan-trimmed viral vaccine (e.g., HIV-1 UFO trimer vaccine) compositions of the invention or pharmaceutical compositions of the invention can be provided as components of a kit. Optionally, such a kit includes additional components including packaging, instructions and various other reagents, such as buffers, substrates, antibodies or ligands, such as control antibodies or ligands, and detection reagents. An optional instruction sheet can be additionally provided in the kits.

### EXAMPLES

The following examples are offered to illustrate, but not to limit the present invention.

### Example 1 Endo-H Treatment of BG505 UFO Trimer-Presenting SApNPs

Two HIV-1 BG505 UFO trimer-presenting nanoparticle vaccine constructs were expressed and purified for glycan trimming studies. One of the SApNPs is formed of a polypeptide chain sequence containing from the N-terminus to the C-terminus: BG505 UFO trimer (SEQ ID NO:4), E2p nanoparticle subunit sequence (SEQ ID NO:1), a locking domain subunit sequence (SEQ ID NO:5), and a T-cell epitope (SEQ ID NO:7). The second SApNP is formed of a polypeptide chain sequence containing from the N-terminus to the C-terminus: BG505 UFO trimer (SEQ ID NO:4), a GS rich linker sequence (SEQ ID NO:8), 13-01 variant nanoparticle subunit sequence (SEQ ID NO:2), a locking domain subunit sequence (SEQ ID NO:6), and a T-cell epitope (SEQ ID NO:7). More detailed information about the structure, construction and activities of these HIV-1 UFO-timer nanoparticle vaccine constructs are described in the art, e.g., WO2019/241483.

The BG505 UFO trimer-presenting SApNPs can be produced in various CHO cells. In laboratory-scale production, they can be transiently expressed in ExpiCHO cells (Thermo Fisher). In large-scale production, they can be either transiently expressed in CHO—S cells or stably expressed in CHO-K1 cells. Immunoaffinity chromatography (IAC) columns based on human antibodies PGT145 and 2G12 was used to purify SApNPs from the CHO cell supernatant, followed by size-exclusion chromatography (SEC) on a Superose 6 column with other polishing steps.

The purified SApNP material is subjected to glycan-trimming by Endo-H, which is followed by SEC purification on a Superose 6 column with other polishing steps. The Endo-H treatment is performed according to the manufacturer's instructions (New England BioLabs) with minor changes. Specifically, the treatment is conducted at room temperature (25° C.) using purified SApNP material without denaturing for 4 hr. The maximum SApNP vs. endo-H ratio (100 μg vs. 12,500 units or 25 μl) in the manual is used to ensure the most complete enzymatic digestion of N-linked glycans on the surface.

### Example 2 Purification and Characterization of Endo-H-Treated SApNPs

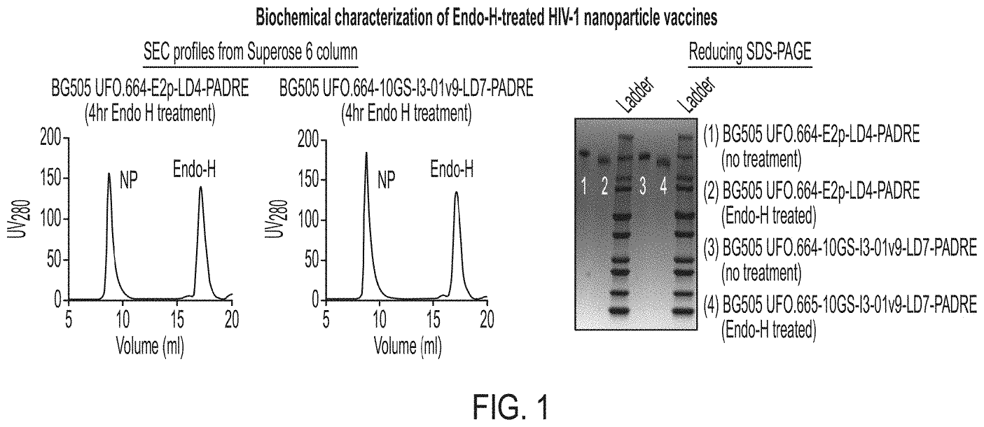

Following Endo-H treatment, glycan-trimmed SApNPs can be purified from the SApNP/Endo-H/glycan mix by SEC on a Superose 6 column. In a recent study, we processed the CHO-K1-produced SApNPs with Endo-H and purified the glycan-trimmed SApNPs using a Superose 6 column (FIG. 1, left). Due to the large size and high molecular weight, the first elution peak corresponded to glycan-trimmed SApNPs, with a tailing peak corresponding to Endo-H. We then characterized the glycan-trimmed SApNPs by SDS-PAGE under the reducing conditions (FIG. 1, right). Compared with the wildtype SApNPs, the glycan-trimmed SApNPs showed a downshifted band on the SDS-PAGE gel, suggesting a reduction in molecular weight due to glycan trimming by Endo-H. Overall, we observed comparable SEC profiles and SDS-PAGE gels for both E2p and I3-01v9-based SApNPs, suggesting a similar outcome due to Endo-H treatment.

### Example 3 Site-Specific Glycan Profiles of Endo-H-Treated SApNPs

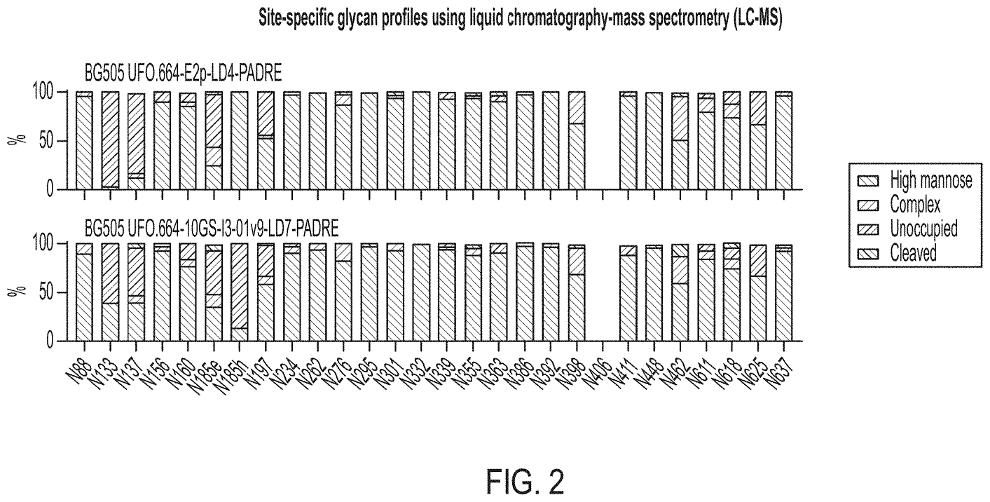

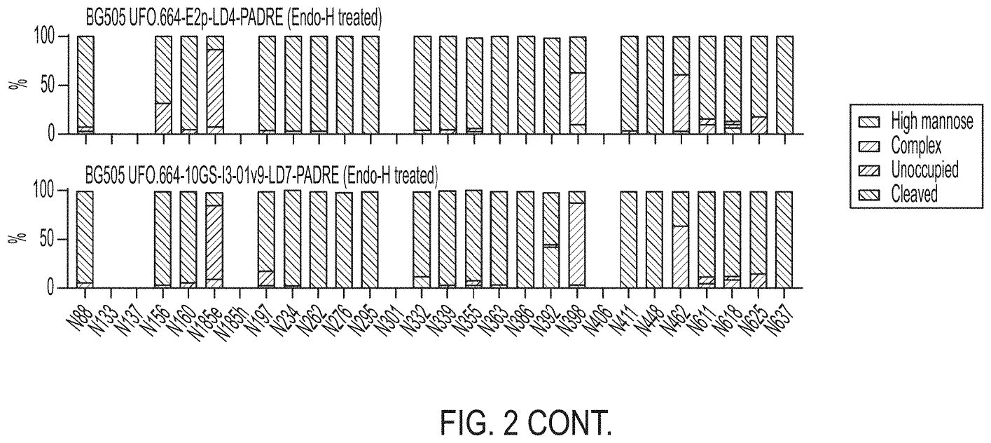

Although a lower band was observed for the Endo-H-treated SApNPs in SDS-PAGE, a higher-precision analysis is needed to confirm the trimming effect for all the N-linked glycosylation sites in the BG505 Env sequence. To this end, we determined the site-specific glycosylation and occupancy profiles for both E2p and I3-01v9-based SApNPs, with and without Endo-H treatment (FIG. 2). We digested three aliquots of the given SApNP sample separately with trypsin, chymotrypsin, and alpha lytic protease. This generated peptides and glycopeptides which only contain a single N-linked glycosylation site to allow the site-specific composition and occupancy to be determined. Liquid chromatography-mass spectrometry (LC-MS) was used to analyze glycopeptides on an Orbitrap Eclipse mass spectrometer. Three analytical repeats of each digest performed to understand the variability of the system. Overall, the data was highly reproducible with little deviation in compositions with a few exceptions. This demonstrated that this method of analysis is robust and reproducible on complex SApNP samples. A new category called “cleaved” (cyan) was added. This refers to the abundance of glycans consisting of a single HexNAc, in this case N-acetylglucosamine, which is a signature of Endo-H treatment as only a single monosaccharide remains. Because the majority of N-linked glycosylation sites on SApNPs are oligomannose-type glycans (green) and these glycans are very sensitive to Endo-H treatment, glycan trimming appeared to have been very efficient at most sites, with the oligomannose glycans being cleaved and complex-type glycans being resistant, e.g., N398 and N462. Overall, we observed similar site-specific glycan profiles for E2p and I3-01v9-based SApNPs, suggesting highly efficient trimming by Endo-H at most sites with oligomannose-type glycans.

### Example 4 Structural and Antigenic Profiles of Endo-H-Treated SApNPs

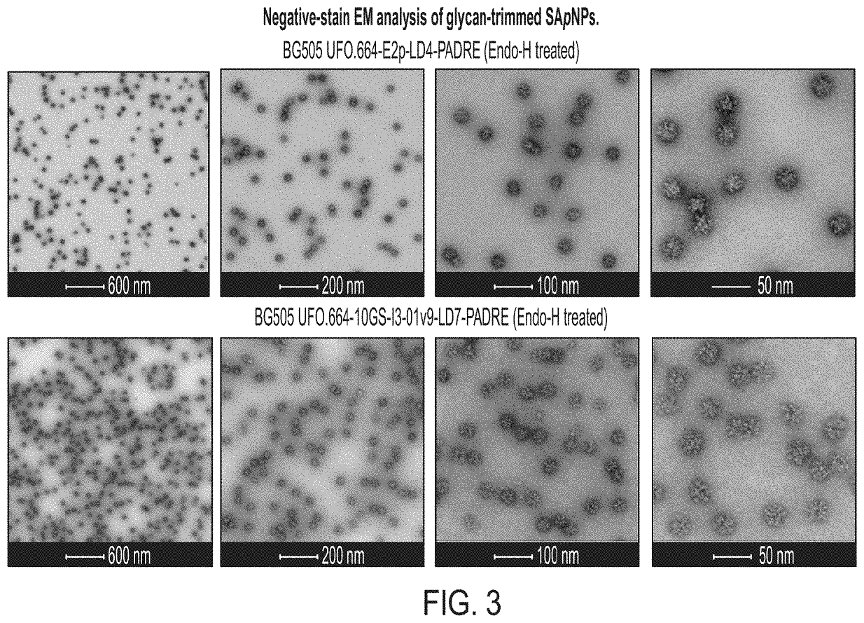

Because the particulate structure of SApNP vaccines is the key to their superior immune response, whether Endo-H treatment has any adverse effect on the structural stability of SApNP must be investigated. To this end, we performed negative-stain electron microscopy (ns-EM) analysis on two SApNP vaccines following Endo-H treatment and SEC purification in several repeated experiments (FIG. 3). Exceptional purity was observed in all ns-EM experiments: large trimer-presenting particles were visible in the EM micrographs, without any contaminant species or unassembled species in the image. The image quality obtained for the Endo-H treated SApNPs was comparable to or greater than the image quality obtained for SApNPs with full-length glycans. We also noticed that the BG505 Env trimer structures become more clearly defined in the EM images of glycan-trimmed SApNPs, supporting the notion that protein structure would be more exposed after cutting flexible surface glycans near the base groups attached to an Asparagine residue.

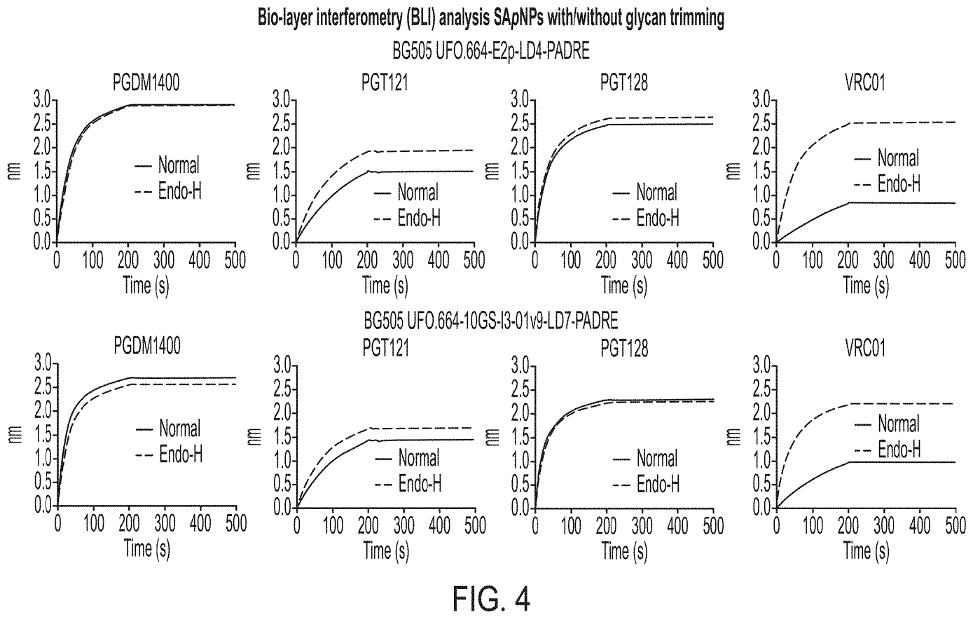

Following structural characterization by ns-EM, we assessed the antigenicity of glycan-trimmed SApNPs by bio-layer interferometry (BLI) on an Octet RED96 instrument using representative human neutralizing antibodies (FIG. 4). These antibodies target the V1V2 apex (PGDM1400), the N332 supersite (PGT121 and PGT128), and the CD4bs (VRC01). Notably, PGDM1400, PGT121, and PGT128 are glycan-reactive antibodies that interact with both protein backbone and Env glycans. PGDM1440 is a somatic variant of PGT145 that interacts with Mans at N160, whereas PGT121 and PGT128 require Man_{8/9} at N332 and other glycans nearby. Remarkably, the Endo-H treated SApNPs bound to glycan-reactive human antibodies at the same level as did the wildtype SApNPs with full-length glycans but showed much higher binding affinity for VRC01 (2 to 3.5-fold). This suggests that glycan trimming by Endo-H can significantly improve the antibody access to the CD4bs without negatively affecting antibody recognition of key glycan epitopes.

### Example 5 Immunogenicity of Endo-H-Treated SApNPs in Wildtype Mice

We performed a mouse study to determine the immunogenicity of glycan-trimmed BG505 UFO trimer-presenting SApNPs. Briefly, 4 groups of BALB/c mice, 8 mice per group, were immunized with E2p and I3-01v9-based SApNPs with 3-week intervals. Two prime-boost regimens were tested. In regimen #1 (homologous), mice received 4 doses of glycan-trimmed SApNP, whereas in regimen #2 (heterologous), mice were primed with 2 doses of glycan-trimmed SApNP and then boosted with 2 doses of wildtype SApNP. In this study, the E2p-based SApNP is formulated with AddaVax, an oil-in-water type of adjuvant, whereas the I3-01v9-based SApNP is mixed with aluminum phosphate. Serum samples before immunization and 2 weeks after each dose were collected for antigen binding assays based on enzyme-linked immunoassay (ELISA) and for neutralization assays after IgG purification.

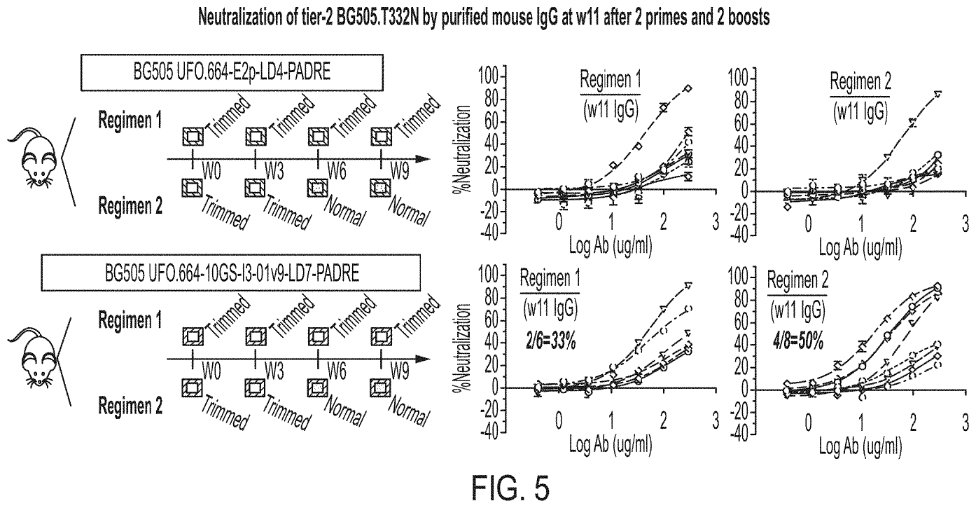

After sample collection, purified mouse IgG were tested in TZM-bl pseudovirus neutralization assays against tier-2 BG505.T332N, other tier-2 isolates in the global virus panel, and selected tier-1 isolates. Specifically, mouse IgGs from the last time point (week 11) were tested against BG505.T332N, with a 300 μg/ml starting concentration followed by a series of 3-fold dilutions (FIG. 5). The results indicate that several animals in each group have generated tier-2 neutralizing antibodies. The 13-01v9-based SApNP appeared to outperform the E2p-based SApNP in terms of both potency and the frequency of vaccine responders. While four injections of glycan-trimmed I3-01v9 SApNP resulted in potent neutralizing responses in 2 of 6 mice (FVR=33%), two primes with glycan-trimmed I3-01v9 SApNP followed by two boosts with wildtype I3-01v9 SApNP generated potent neutralizing responses in 4 of 8 mice (FVR=50%). Such robust neutralizing antibody response has not been observed for wildtype SApNP immunization in our previous studies. The results also suggest that a heterologous prime-boost strategy may be most effective at eliciting such responses.

### Example 6 Endo-H Treatment of HIV-1 BG505 UFO Trimer

The BG505 UFO trimer can be produced in various CHO cells. In laboratory-scale production, this trimer can be transiently expressed in ExpiCHO cells (Thermo Fisher). In large-scale production, this trimer can be either transiently expressed in CHO-S cells (Thermo Fisher) or stably expressed in CHO-K1 cells. We used immunoaffinity chromatography (IAC) columns based on human antibodies PGT145 and 2G12 to purify the BG505 UFO trimer from the CHO cell supernatant, followed by size-exclusion chromatography (SEC) on a Superdex 200 10/300 column with other polishing steps. The purified trimer was subjected to glycan trimming by Endo-H, which is followed by SEC purification on a Superdex 200 10/300 column with other polishing steps. A fusion protein of Endo-H and Maltose-binding protein (MBP), Endo-Hf (New England BioLabs), was used here to facilitate the enzyme removal at a later stage. The Endo-H treatment was performed according to the manufacturer's instructions (New England BioLabs) with minor changes. Specifically, the treatment was carried out at room temperature (25° C.) using SEC-purified trimer material without denaturing for 4 hr. The maximum trimer vs. endo-H ratio (100 μg vs. 12,500 units or 25 μl) in the manual was used to ensure the most complete enzymatic digestion of N-linked glycans on the trimer surface. This glycan trimming protocol can be used for trimming UFO trimers derived from other HIV-1 Envs proteins.

### Example 7 Purification and Characterization of Endo-H-Treated BG505 UFO Trimer

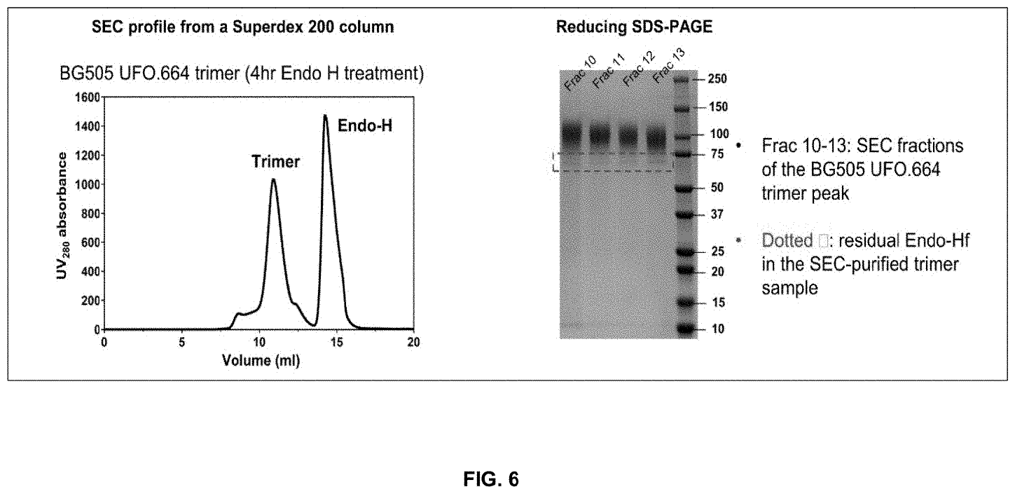

After Endo-H treatment, glycan-trimmed UFO trimers can be purified from the trimer/Endo-Hf/glycan mix first by another round of SEC. In this study, we enzymatically processed the ExpiCHO-produced BG505 UFO trimer with Endo-Hf and purified the glycan-trimmed trimer using a Superdex 200 10/300 column (FIG. 6, left). The elution peaks of the BG505 UFO trimer and Endo-Hf were separate but very close to one another in the SEC profile due to their similar size. We analyzed the SEC fractions within the trimer peak by SDS-PAGE under the reducing conditions (FIG. 6, right). A light band was observed for all fractions, suggesting residual Endo-Hf in the trimer sample that must be removed in further polishing steps.

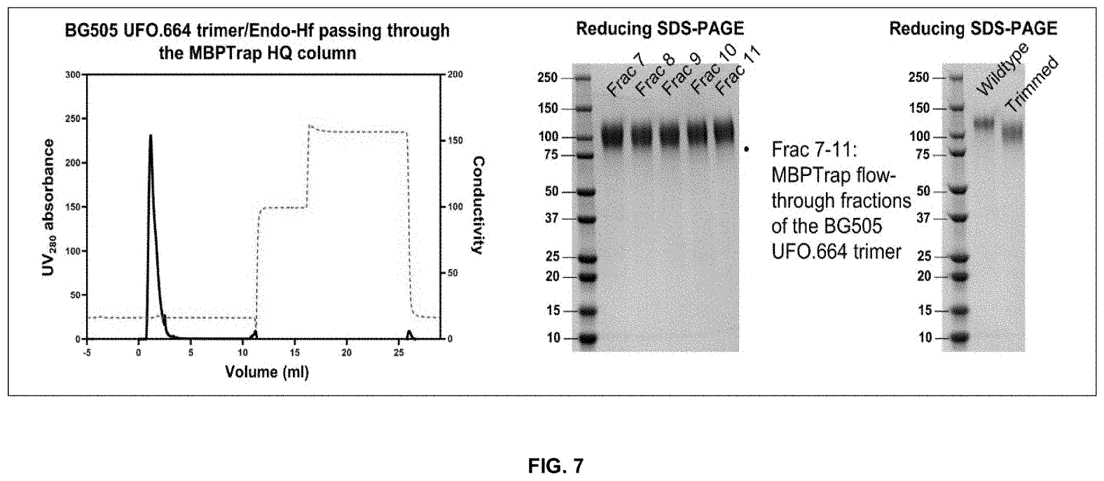

We then processed the Endo-H-treated, SEC-purified BG505 UFO trimer using a Cytiva MBPTrap HQ column (FIG. 7, left). Residual Endo-Hf within the trimer sample was trapped in the column, whereas the BG505 UFO trimer flew through (FIG. 7, left, the 1^{st} peak). The MBP-tagged Endo-H was washed off the column using 0.5 M NaOH and could be recognized on the profile (FIG. 7, left, the 2^{nd} low peak). The flow-through fractions were collected as the purified BG505 UFO trimer for analysis by SDS-PAGE under reducing conditions (FIG. 7, middle). Only trimer bands were observed on the gel, suggesting that residual Endo-Hf had been removed. An anti-MBP mouse antibody was used in ELISA to detect whether the Endo-H-treated SEC/MBPTrap-purified trimer still contains Endo-H, with an untreated trimer sample included as a control. The resulting OD450 readouts were 0.055 and 0.060 for the two samples, confirming that the residual Endo-Hf had been thoroughly removed. On the SDS-PAGE gel, the glycan-trimmed trimer showed a lower band compared with the wildtype trimer (FIG. 7, right), suggesting a reduction in molecular weight (MW) due to the shortening of glycan chains in Endo-H treatment.

### Example 8 Structural Analysis of the Glycan-Trimmed BG505 UFO Trimer

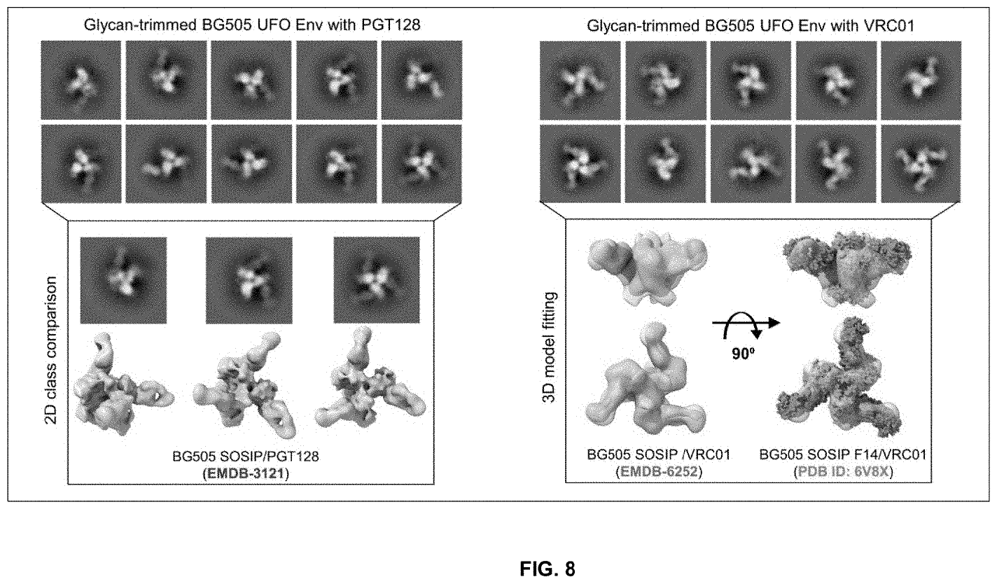

We then investigated whether Endo-H treatment affects the trimer structure and antibody recognition using negative-stain electron microscopy (ns-EM) (FIG. 8). Broadly neutralizing antibody PGT128 binds the N332 supersite at the V3 base and interacts with Man₈₋₉. The removal of all mannose groups at the N295, N301, and N332 sites may destabilize PGT128 and affect its angle of approach. EM images showed that one Endo-H-treated BG505 UFO trimer could bind to one to three PGT128 Fabs, with less resolved density for the bound Fabs in the 2D averages (FIG. 8, left). Comparison with the cryo-EM model of the BG505 SOSIP/PGT128 complex (EMDB-3121) confirmed that PGT128, albeit increased flexibility, still bound to the same epitope on the Env. Broadly neutralizing antibody VRC01 targets the CD4 binding site (CD4bs) protected by a ring of glycans with the N276 glycan positioned as a critical barrier to the development of VRC01-class antibodies. Trimming the glycan shield should in principle improve the CD4bs recognition by VRC01. Indeed, we obtained a 12.4 Å-resolution 3D model of the BG505 UFO/VRC01 complex, which fit well with the previous EM models of BG505 SOSIP/VRC01 complexes (FIG. 8, right).

### Example 9 Antigenic Profiling of Endo-H-Treated BG505 UFO Trimer by BLI

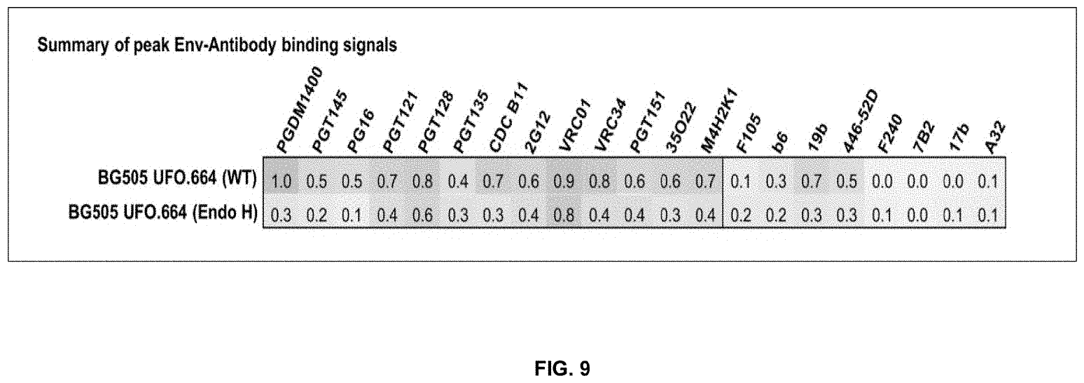

We then used analytical sensors in bio-layer interferometry (BLI) analysis to measure the trimer binding to a panel of HIV-1 antibodies, including 13 neutralizing antibodies and 8 non-neutralizing antibodies. The results indicate that overall the wildtype BG505 UFO trimer preferentially bound to neutralizing antibodies but not the non-neutralizing antibodies with the exception of 19b and 446-52D, which target the immunodominant V3 tip. Endo-H treatment notably reduced the trimer binding to neutralizing antibodies directed to the V2 apex, such as PGDM1400, PGT145, and PG16. Surprisingly, Endo-H treatment had a limited impact on the broadly neutralizing antibody PGT128, which recognizes the N332 supersite by interacting with the ‘GDIR’ motif and high-mannose (Man₈₋₉) glycans at N332 and N301. Endo H treatment retained the accessibility of the CD4bs to broadly neutralizing antibody VRC01, upon the trimming of the glycan barrier surrounding this protein epitope. The peak antibody binding signals were summarized for the convenience of comparison (FIG. 9). Overall, Endo H reduced trimer binding to glycan-reactive neutralizing antibodies with differential effects.

### Example 10 Immunogenicity of Endo-H-Treated BG505 UFO Trimer in Wildtype Mice

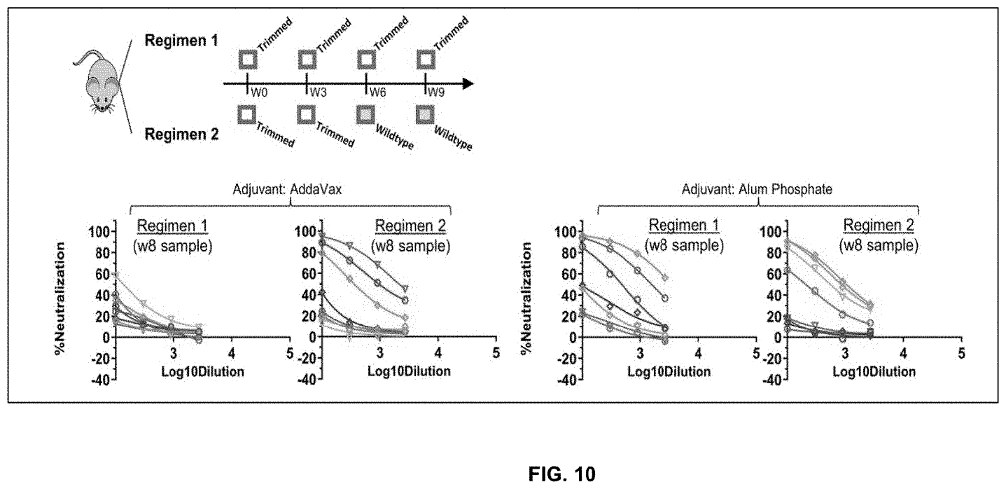

We further performed a mouse study to determine the immunogenicity of glycan-trimmed BG505 UFO trimer. Briefly, 4 groups of BALB/c mice, 8 mice per group, were immunized with the BG505 UFO trimer with 3-week intervals. Two prime-boost regimens were tested. In regimen 1 (homologous), mice received 4 doses of glycan-trimmed trimer, whereas in regimen 2 (heterologous), mice were primed with 2 doses of glycan-trimmed trimer and then boosted with 2 doses of wildtype trimer (FIG. 10, top). The trimer protein was either formulated with AddaVax, an oil-in-water type of adjuvant, or with aluminum phosphate (AP). Serum samples before immunization and 2 weeks after each dose were analyzed in TZM-bl pseudovirus neutralization assays against autologous tier 2 BG505.T332N. In the study, the week-8 sera after 3 does were tested against BG505.T332N, with a 100-fold starting dilution followed by a series of 3-fold dilutions (FIG. 10, bottom). With AddaVax, regimen 1 did not show any positive hits, whereas regimen 2 had three mice showing >50% neutralization at the 1^{st} dilution. With AP, a stronger neutralizing response was observed. Regardless of the regimen used, three mice showed >50% neutralization at the first dilution, with an additional mouse reaching ˜40% in the regimen 2 group. These data indicate that glycan-trimmed trimer can be used with an alum adjuvant in a heterologous prime-boost strategy to achieve a stronger response.

The invention thus has been disclosed broadly and illustrated in reference to representative embodiments described above. It is understood that various modifications can be made to the present invention without departing from the spirit and scope thereof.

It is further noted that all publications, sequence accession numbers, patents and patent applications cited herein are hereby expressly incorporated by reference in their entirety and for all purposes as if each is individually so denoted. Definitions that are contained in text incorporated by reference are excluded to the extent that they contradict definitions in this disclosure.

What is claimed is:

1. A method to enhance immunogenicity or responder frequency of a vaccine comprising a glycan shielded immunogenic protein or a variant thereof from a virus, comprising (a) contacting the vaccine with an enzyme that is capable of removing or shortening the N-linked glycan chain from the protein, or (b) expressing a polynucleotide sequence encoding the protein or variant thereof in a cell line that produces short glycans and/or lacks N-acetylglucosaminyltransferase I; thereby enhancing immunogenicity or responder frequency of the vaccine.

2. The method of claim 1, wherein the virus is HIV-1, Lassa virus, hepatitis C virus, influenza virus, or SARS-CoV-2.

3. The method of claim 1, wherein the entire length of the N-linked glycan chain is trimmed.

4. The method of claim 1, wherein length of the N-linked glycan chain is trimmed by about 50%, about 60%, about 70%, about 80%, or about 90%.

5. The method of claim 1, wherein the enzyme is an endoglycosidase (Endo) or a peptide/N-glycosidase.

6. The method of claim 1, wherein the enzyme is endoglycosidase H (Endo-H), F1 (Endo-F1), F2 (Endo-F2), or F3 (Endo-F3).

7. The method of claim 1, wherein the immunogenic protein is a modified HIV-1 envelope gp140 protein comprising a gp120 polypeptide and a soluble gp41 polypeptide, wherein the N-terminus of heptad 1 region (HR1) of the gp41 polypeptide is replaced with a loop sequence of about 6 to about 14 amino acid residues in length that stabilizes the pre-fusion gp140 structure.

8. The method of claim 7, wherein the modified HIV-1 envelope gp140 protein is an uncleaved prefusion-optimized (UFO) gp140 trimer.

9. The method of claim 8, wherein the modified HIV-1 envelope gp140 protein is a chimeric trimer comprising a modified gp41_{ECTO} domain from HIV-1 strain BG505.

10. The method of claim 8, wherein the modified HIV-1 envelope gp140 protein further comprises a flexible linker sequence that substitutes for the cleavage site sequence between gp120 and gp41.

11. The method of claim 10, wherein the linker sequence comprises (G₄S)₂ (SEQ ID NO:8) and substitutes for residues 501-518 at the cleavage site, and wherein numbering of the amino acid residues corresponds to that of HIV-1 strain BG505 SOSIP.664 gp140.

12. The method of claim 11, wherein the modified HIV-1 envelope gp140 protein further comprises (a) an engineered disulfide bond between gp120 and gp41 and/or (b) a stabilizing mutation in gp41.

13. The method of claim 12, wherein the engineered disulfide bond is between residues A501C and T605C, and the stabilizing mutation is I559P.

14. The method of claim 9, wherein the N-terminus of heptad 1 region (HR1) in the gp41_{ECTO} polypeptide is replaced with a loop sequence NPDWLPDM (SEQ ID NO:9).

15. The method of claim 14, further comprising (a) a linker sequence (G₄S)₂ (SEQ ID NO:8) that substitutes for residues 508-511 at the cleavage site, and (b) an engineered disulfide bond between residues A501C and T605C; wherein numbering of the amino acid residues corresponds to that of HIV-1 strain BG505 SOSIP.664 gp140.

16. The method of claim 5, wherein the vaccine is contacted with the enzyme at room temperature (25° C.) without denaturing for 4 hr.

17. The method of claim 5, wherein the enzyme vs protein ratio is sufficient for complete enzymatic digestion of N-linked glycans on the immunogenic protein or variant thereof.

18. The method of claim 1, further comprising purification of the enzyme treated vaccine.

19. The method of claim 8, wherein the subunit sequence of the UFO gp140 trimer comprises the sequence shown in SEQ ID NO:4, a conservatively modified variant or a substantially identical sequence thereof.

20. An HIV-1 vaccine, produced by a process comprising the steps of (1) expressing a polynucleotide encoding the subunit of a modified HIV-1 Env gp140 trimer protein, and (2) trimming N-glycosylation chain on the expressed protein with an enzyme.

21. The HIV-1 vaccine of claim 20, wherein the enzyme is endoglycosidase H (Endo-H).

22. The HIV-1 vaccine of claim 20, wherein the modified HIV-1 envelope trimer gp140 protein is an uncleaved prefusion-optimized (UFO) gp140 trimer.

23. A pharmaceutical composition, comprising the vaccine of claim 20 and a pharmaceutically acceptable carrier.

24. A method of treating or preventing HIV-1 infection in a subject, comprising administering to the subject a pharmaceutical composition comprising a therapeutically effective amount of the HIV-1 vaccine of claim 20, thereby treating or preventing HIV-1 infection in the subject.
